SEQUENCE LISTING (26 sequences)

SEQ ID NO: 1 - DNA, 20 bp, Artificial sequence
  Synthetic primer
attgtcgtcgtgcctcgaat

SEQ ID NO: 2 - DNA, 20 bp, Artificial sequence
  Synthetic primer
aaaccctttccacctacccg

SEQ ID NO: 3 - DNA, 21 bp, Artificial sequence
  Synthetic primer
cacccaggcaaatgtttggtc

SEQ ID NO: 4 - DNA, 20 bp, Artificial sequence
  Synthetic primer
gcttagccttgaggagcgag

SEQ ID NO: 5 - DNA, 21 bp, Artificial sequence
  Synthetic primer
ctgttgctgagctaccaagga

SEQ ID NO: 6 - DNA, 20 bp, Artificial sequence
  Synthetic primer
tcatcaggcaaccaagccat

SEQ ID NO: 7 - DNA, 20 bp, Artificial sequence
  Synthetic primer
gtggctccctacaccaaaca

SEQ ID NO: 8 - DNA, 20 bp, Artificial sequence
  Synthetic primer
gccagaagcccagtggtaat

SEQ ID NO: 9 - DNA, 20 bp, Artificial sequence
  Synthetic primer
attatgtcgccggaactggt

SEQ ID NO: 10 - DNA, 20 bp, Artificial sequence
  Synthetic primer
agaccgaggaagacctgact

SEQ ID NO: 11 - DNA, 20 bp, Artificial sequence
  Synthetic primer
tggttccccagttctgttgg

SEQ ID NO: 12 - DNA, 22 bp, Artificial sequence
  Synthetic primer
cctggacatgtctggaaagtga

SEQ ID NO: 13 - DNA, 22 bp, Artificial sequence
  Synthetic primer
ttaccagcttacagactcctgc

SEQ ID NO: 14 - DNA, 22 bp, Artificial sequence
  Synthetic primer
tgtgggagttccctgagtctaa

SEQ ID NO: 15 - DNA, 20 bp, Artificial sequence
  Synthetic primer
catgccaacacccgctcata

SEQ ID NO: 16 - DNA, 20 bp, Artificial sequence
  Synthetic primer
ccccaacggggttgtcaaaa

SEQ ID NO: 17 - DNA, 22 bp, Artificial sequence
  Synthetic primer
cacacaagccagcttttactgt

SEQ ID NO: 18 - DNA, 20 bp, Artificial sequence
  Synthetic primer
acttggtaggggcacattgg

SEQ ID NO: 19 - DNA, 20 bp, Artificial sequence
  Synthetic primer
aaaatgttctcccgccagca

SEQ ID NO: 20 - DNA, 20 bp, Artificial sequence
  Synthetic primer
caagcatgcccagtgtttgc

SEQ ID NO: 21 - DNA, 20 bp, Artificial sequence
  Synthetic primer
cagcgactctgaggaggaac

SEQ ID NO: 22 - DNA, 20 bp, Artificial sequence
  Synthetic primer
gctggtgcattttcggttgt

SEQ ID NO: 23 - DNA, 20 bp, Artificial sequence
  Synthetic primer
ggcttatgtgatcgctgtgc

SEQ ID NO: 24 - DNA, 20 bp, Artificial sequence
  Synthetic primer
atgtgctttggccattggag

SEQ ID NO: 25 - DNA, 45 bp, Artificial sequence
  Synthetic primer
ttccgctacattccacccaacgctccagagaatttttgggaagtt

SEQ ID NO: 26 - DNA, 45 bp, Artificial sequence
  Synthetic primer
aacttcccaaaaattctctggagcgttgggtggaatgtagcggaa

(19) United States
(12) Patent Application Publication
(10) Pub. No.: US 2022/0047596 A1
(43) Pub. Date: Feb. 17, 2022

(54) COMBINATION OF PARP INHIBITOR AND BRD4 INHIBITOR FOR THE TREATMENT OF CANCER

(71) Applicant: Board of Regents, The University of Texas System, Austin, TX (US)

(72) Inventors: Gordon B. MILLS, Houston, TX (US); Chaoyang SUN, Houston, TX (US)

(73) Assignee: Board of Regents, The University of Texas System, Austin, TX (US)

(21) Appl. No.: 17/275,604

(22) PCT Filed: Sep. 12, 2019
(86) PCT No.: PCT/US2019/050887

§ 371 Date: Mar. 11, 2021

Related U.S. Application Data
(60) Provisional application No. 62/730,171, filed on Sep. 12, 2018.
(60) Provisional application No. 62/730,171, filed on Sep. 12, 2018.

(51) Int. Cl. A61K 31/5025; A61K 31/551; A61K 45/06; A61P 35/00

(52) U.S. Cl. CPC A61K 31/5025; A61P 35/00; A61K 45/06; A61K 31/551

(57) ABSTRACT

Provided herein are methods of treating cancer comprising administering a PARP inhibitor which may be combined with a BRD4 inhibitor. In one embodiment, the present disclosure provides a method for treating cancer in a subject comprising administering an effective amount of a poly-ADP-ribose polymerase (PARP) inhibitor in combination with a bromodomain-containing protein 4 (BRD4) inhibitor to the subject. In some aspects, the administration of the PARP inhibitor and BRD4 inhibitor results in greater reduction in tumor growth or greater reduction in tumor mass relative to administration of PARP inhibitor or BRD4 inhibitor alone.

This application claims the benefit of U.S. Provisional Patent Application No. 62/730,171, filed Sep. 12, 2018, the entirety of which is incorporated herein by reference.

### INCORPORATION OF SEQUENCE LISTING

The sequence listing that is contained in the file named “UTFCP1379WO_ST25.txt”, which is 6 KB (as measured in Microsoft Windows®) and was created on Sep. 12, 2019, is filed herewith by electronic submission and is incorporated by reference herein.

### BACKGROUND

### 1. Field

The present invention relates generally to the fields of medicine and immunology. More particularly, it concerns the combination therapy of PARP and BRD4 inhibition for cancer therapy.

### 2. Description of Related Art

DNA double-strand breaks (DSBs) can lead to mutation, chromosomal aberration, or cell death. DSBs are repaired by two main mechanisms: non-homologous end joining (NHEJ) and homologous recombination (HR) (Hoeijmakers, 2001; Jackson and Bartek, 2009). Mutation-prone NHEJ ligates broken DNA ends without requiring sequence complementarity. In contrast, HR mediates high fidelity DNA repair using sister chromatids as the repair template. The different DSB repair pathways are tightly controlled (Huertas, 2010). HR is instigated by DSB end resection, which generates a long single-stranded DNA (ssDNA) that is protected by replication protein A (RPA) (Broderick et al., 2016; Kaidi et al., 2010). C-terminal binding protein (CtBP) interacting protein (CtIP) physically interacts with the MRE11-RAD50-NBS1 (MRN) complex at DSBs, promoting DNA end resection, ssDNA generation, and nuclease activity of the MRN complex (Davies et al., 2015; Yun and Hiom, 2009).

CtIP downregulation abolishes ssDNA formation, and impairs HR function (Sartori et al., 2007; Yun and Hiom, 2009). Bromodomain containing 4 (BRD4), a member of the bromo-domain and extraterminal (BET) protein family, maintains and facilitates oncogenic transcription directly by recruiting transcriptional machinery or indirectly by binding to enhancers, contributing to cancer cell proliferation and survival (Yang et al., 2005). BRD4 can be selectively targeted with small-molecule inhibitors, such as JQ1 (Filippakopoulos et al., 2010), GSK1210151A (I-BET151 [Dawson et al., 2011]), GSK525762A (I-BET-762 [Nicodeme et al., 2010]), GSK1324726A (I-BET-726 [Gosmini et al., 2014]), and AZD5153 (Rhyasen et al., 2016). BRD4i are active in preclinical models of hematological malignancies and solid tumors (Asangani et al., 2014; Delmore et al., 2011; Filippakopoulos et al., 2010; Yokoyama et al., 2016). Multiple BRD4i have entered clinical trials (NCT01587703, NCT03059147, NCT02419417, NCT01949883, NCT03068351, and NCT02259114).

BRD4 is frequently amplified and correlates with poor prognosis in patients with high-grade serous ovarian carcinoma (HGSOC) (Zhang et al., 2016). In addition, at least half of HGSOCs exhibit aberrations in the HR pathway (Cancer Genome Atlas Research Network, 2011). Tumor cells that lack functional BRCA1, BRCA2, or other key components of the HR pathway, are highly sensitivity to poly(ADP-ribose) polymerase inhibitor (PARPi) (Bryant et al., 2005; Ledermann et al., 2016), leading to regulatory approval of three different PARPi for ovarian cancer treatment (Kaufman et al., 2015; Mirza et al., 2016; Swisher et al., 2017). Although high response rates are achieved, most tumors rapidly become resistant, including BRCA1/2 mutant cancers. Therefore, the development of strategies to prevent or reverse PARPi resistance to increase the duration of response and expand the utility of PARPi to HR-competent tumors is needed.

### SUMMARY

In one embodiment, the present disclosure provides a method for treating cancer in a subject comprising administering an effective amount of a poly-ADP-ribose polymerase (PARP) inhibitor in combination with a bromodomain-containing protein 4 (BRD4) inhibitor to the subject. In some aspects, the administration of the PARP inhibitor and BRD4 inhibitor results in greater reduction in tumor growth or greater reduction in tumor mass relative to administration of PARP inhibitor or BRD4 inhibitor alone. In certain aspects, the subject is human.

In some aspects, the subject is PARP inhibitor resistant. In other aspects, the subject is PARP inhibitor sensitive. In particular aspects, the administration of the PARP inhibitor in combination with the BRD4 inhibitor prevents emergence of PARP inhibitor resistance.

In certain aspects, the cancer is a RAS/BRAF, BRCA1/2, and/or p53 mutant cancer. In some aspects, the RAS/BRAF mutation is KRAS or NRAS. In particular aspects, the cancer is homologous recombination (HR) competent. In specific aspects, the HR competent cancer is a RAS/BRAF, BRCA1/2, and/or p53 wild-type cancer. In some aspects, the cancer is breast cancer, ovarian cancer, pancreatic cancer, colorectal cancer, lung cancer, or melanoma. In some aspects, the subject has increased expression of C-terminal binding protein interacting protein (CtIP).

In certain aspects, the PARP inhibitor is Olaparib, BMN673, Niraparib, Rucaparib, or ABT888 (Veliparab). In particular aspects, the PARP inhibitor is BMN673. In some aspects, the BRD4 inhibitor is JQ1, GSK1210151A (I-BET151), GSK1324726A (I-BET-726), or AZD5153. In particular aspects, the BRD4 inhibitor is JQ1.

In some aspects, the PARP inhibitor and/or BRD4 inhibitor are administered orally, intravenously, intraperitoneally, intratracheally, intratumorally, intramuscularly, endoscopically, intralesionally, percutaneously, subcutaneously, regionally, or by direct injection or perfusion. In particular aspects, the PARP inhibitor and/or BRD4 inhibitor are administered orally. In some aspects, the PARP inhibitor is administered at a dose of 200-400 mg/day. In certain aspects, the BRD4 inhibitor is administered at a dose of 10-40 mg/day. In some aspects, the PARP inhibitor and BRD4 inhibitor are administered more than once. In particular aspects, the PARP inhibitor and BRD4 inhibitor are administered daily. In some aspects, the PARP inhibitor and BRD4 inhibitor are administered concurrently. In certain aspects, the PARP inhibitor is administered before the BRD4 inhibitor. In specific aspects, the BRD4 inhibitor is administered before the PARP inhibitor.

In some aspects, the administration results in induction of homologous repair deficiency. In particular aspects, the induction of homologous repair deficiency results in an increase in DNA damage and checkpoint defects. In some aspects, the administration results decreased expression of WEE1 and/or TOPBP1. In particular aspects, the administration results in decreased expression of C-terminal binding protein interacting protein (CtIP).

In additional aspects, the method further comprises the step of administering at least one additional therapeutic agent to the subject. In some aspects, the subject receives at least one additional type of therapy. In particular aspects, the at least one additional type of therapy is selected from the group consisting of chemotherapy, radiotherapy, targeted therapy, and immunotherapy.

In another embodiment, there is provided a method for treating a PARP-resistant cancer or preventing PARP resistance in a subject comprising administering an effective amount of a BRD4 inhibitor to the subject. In some aspects, BRD4 inhibition resensitizes PARP resistant cells to PARP inhibition.

In additional aspects, the method further comprises administering an effective amount of a PARP inhibitor to the subject.

In some aspects, the cancer is breast cancer, ovarian cancer, pancreatic cancer, colorectal cancer, lung cancer, or melanoma. In some aspects, the subject has increased expression of C-terminal binding protein interacting protein (CtIP).

In particular aspects, the PARP inhibitor is Olaparib, BMN673, Niraparib, Rucaparib, or ABT888 (Veliparab). In some aspects, the BRD4 inhibitor is JQ1, GSK1210151A (I-BET151), GSK1324726A (I-BET-726), or AZD5153. In certain aspects, the PARP inhibitor and/or BRD4 inhibitor are administered orally, intravenously, intraperitoneally, intratracheally, intratumorally, intramuscularly, endoscopically, intralesionally, percutaneously, subcutaneously, regionally, or by direct injection or perfusion. In certain aspects, the PARP inhibitor and/or BRD4 inhibitor are administered intravenously. In some aspects, the PARP inhibitor and BRD4 inhibitor are administered more than once. In certain aspects, the PARP inhibitor and BRD4 inhibitor are administered daily. In some aspects, the PARP inhibitor and BRD4 inhibitor are administered concurrently. In certain aspects, the PARP inhibitor is administered before the BRD4 inhibitor. In some aspects, the BRD4 inhibitor is administered before the PARP inhibitor.

In certain aspects, the administration results decreased expression of WEE1 and/or TOPBP1. In some aspects, the administration results in decreased expression of C-terminal binding protein interacting protein (CtIP).

In additional aspects, the method further comprised the step of administering at least one additional therapeutic agent to the subject. In some aspects, the subject receives at least one additional type of therapy. In certain aspects, the at least one additional type of therapy is selected from the group consisting of chemotherapy, radiotherapy, and immunotherapy.

Another embodiment provides a method of predicting response to a PARP inhibitor comprising measuring the expression of CtIP in said subject, wherein low CtIP expression identifies a PARP sensitive cancer and high CtIP expression identifies a PARP resistant cancer. In some aspects, a subject with the PARP sensitive cancer is administered an effective amount of a PARP inhibitor. In certain aspects, a subject with the PARP resistant cancer is administered an effective amount of a BRD4 inhibitor to induce PARP sensitivity. In additional aspects, the subject is further administered an effective amount of a PARP inhibitor.

In some aspects, the cancer is breast cancer, ovarian cancer, pancreatic cancer, colorectal cancer, lung cancer, or melanoma. In particular aspects, the PARP inhibitor is Olaparib, BMN673, Niraparib, Rucaparib, or ABT888 (Veliparab). In specific aspects, the BRD4 inhibitor is JQ1, GSK1210151A (I-BET151), GSK1324726A (I-BET-726), or AZD5153.

In specific aspects, the PARP inhibitor and/or BRD4 inhibitor are administered orally, intravenously, intraperitoneally, intratracheally, intratumorally, intramuscularly, endoscopically, intralesionally, percutaneously, subcutaneously, regionally, or by direct injection or perfusion. In some aspects, the PARP inhibitor and/or BRD4 inhibitor are administered intravenously. In certain aspects, the PARP inhibitor and BRD4 inhibitor are administered more than once. In particular aspects, the PARP inhibitor and BRD4 inhibitor are administered daily. In some aspects, the PARP inhibitor and BRD4 inhibitor are administered concurrently. In particular aspects, the PARP inhibitor is administered before the BRD4 inhibitor. In some aspects, the BRD4 inhibitor is administered before the PARP inhibitor.

In additional aspects, the method further comprises the step of administering at least one additional therapeutic agent to the subject. In some aspects, the subject receives at least one additional type of therapy. In specific aspects, the at least one additional type of therapy is selected from the group consisting of chemotherapy, radiotherapy, targeted therapy, and immunotherapy.

A further embodiment provides a method of treating cancer in a subject comprising administering a BRD4 inhibitor to the subject, wherein the patient has been determined to be resistant to PARP inhibitors. In some aspects, the cancer is breast cancer, ovarian cancer, pancreatic cancer, colorectal cancer, lung cancer, or melanoma. In particular aspects, the PARP inhibitor is Olaparib, BMN673, Niraparib, Rucaparib, or ABT888 (Veliparab). In specific aspects, he BRD4 inhibitor is JQ1, GSK1210151A (I-BET151), GSK1324726A (I-BET-726), or AZD5153.

In yet another embodiment, there is provided a method of inhibiting CtIP expression in a subject comprising administering an effective amount of BRD4 inhibitor to said subject. In some aspects, the subject has cancer. In particular aspects, the cancer is breast cancer, ovarian cancer, pancreatic cancer, colorectal cancer, non-small cell lung cancer, or melanoma. In some aspects, the BRD4 inhibitor is JQ1, GSK1210151A (I-BET151), GSK1324726A (I-BET-726), or AZD5153. In additional aspects, the method further comprises administering an effective amount of a PARP inhibitor to the subject. In some aspects, the PARP inhibitor is Olaparib, BMN673, Niraparib, Rucaparib, or ABT888 (Veliparab).

Further provided herein is a pharmaceutical composition comprising a PARP inhibitor and a BRD4 inhibitor. Also provided herein is the pharmaceutical composition comprising a PARP inhibitor and a BRD4 inhibitor for use in the treatment of cancer. Further embodiments provide the use of a therapeutically effective amount of a PARP inhibitor and a BRD4 inhibitor for the treatment of cancer. In some aspects, the cancer is breast cancer, ovarian cancer, pancreatic cancer, colorectal cancer, lung cancer, or melanoma. In certain aspects, the BRD4 inhibitor is JQ1, GSK1210151A (I-BET151), GSK1324726A (I-BET-726), or AZD5153. In specific aspects, the PARP inhibitor is Olaparib, BMN673, Niraparib, Rucaparib, or ABT888 (Veliparab).

A further embodiment provides a composition comprising a therapeutically effective amount of a PARP inhibitor and a BRD4 inhibitor for the treatment of cancer in a subject. Also provided herein is the use of a PARP inhibitor and a BRD4 inhibitor in the manufacture of a medicament for the treatment of cancer. In some aspects, the PARP inhibitor is Olaparib, BMN673, Niraparib, Rucaparib, or ABT888 (Veliparab). In specific aspects, the BRD4 inhibitor is JQ1, GSK1210151A (I-BET151), GSK1324726A (I-BET-726), or AZD5153. In some aspects, the cancer is breast cancer, ovarian cancer, pancreatic cancer, colorectal cancer, lung cancer, or melanoma.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D: Effect of BRD4 Inhibition on HR. (A) Heatmap (left) and HRD scores (right) from un-supervised clustering of HRD gene signatures using the GSE29799 dataset. Higher scores represent defective HR. Data represent means±SEM. Statistical significances were determined using Student's t test. (B) Relative HRD score represents change (treated minus control) in HRD scores in the indicated GEO datasets after BRD4 inhibition. The top symbol indicates the method of BRD4 inhibition used. Circle size indicates change in HRD scores, while color indicates −log(p) by Student's t test. (C) U2OS DR-GFP cells were treated with 100 nM JQ1 or 100 nM AZD5153 for 24 hr (upper), or transfected with control or BRD4 siRNA for 24 hr (lower), and then transfected with I-Sce1 endonuclease for 48 hr. HR efficiency of treated cells was compared with DMSO or control siRNA, respectively, based on the percentage of GFP+ cells detected by flow cytometry. Data represent mean±SEM of three independent experiments. Student's t test: ***p<0.001. (D) Results of Ingenuity Pathway Analysis (IPA) of pathways significantly altered by BRD4 inhibition in the indicated GEO datasets. Symbol of intervention is as in (B). Numbers in the box correspond to −log(p) calculated by Benjamin-Hochberg method.

FIGS. 2A-2J: Effect of BRD4 Inhibition on CtIP Expression. (A) Heatmap of RPPA data representing “rank-ordered” changes induced by BRD4i treatment. Proteins with consistent decreases are on the left and increases are on the right of the heatmap. Statistically significant changes (Z scores) indicated in boxes. (B) Western blot of indicated proteins in HOC1 cells treated with the indicated dose of JQ1 for 48 hr (left) or treated with 200 nM JQ1 for the indicated length (right). (C) Western blot of indicated proteins in HOC1 cells after BRD4 silencing for 48 hr. (D) Western blot of indicated proteins in HOC1 cells treated with 200 nM JQ1 or 200 nM GSK1324726A for 48 hr. (E) Correlation between BRD4 and CtIP protein expression in MCLP database. (F) Representative image of IHC with BRD4 or CtIP antibody (left) and correlation between BRD4 and CtIP expression by IHC (right) in ovarian cancer tissues. Scale bar, 25 mm (G) Correlation between BRD4 and CtIP protein expression in NCI60 dataset. (H) IPA with genes in CtIP coexpression signature. (I) Gene set enrichment analysis (GSEA) plot of the CtIP coexpression signature in GSE29799 after BRD4 inhibition. (J) GSEA plot of enrichment score (ES) of CtIP coexpression signature in indicated GEO datasets after BRD4 inhibition. Symbol of intervention is as in FIG. 1B.

FIGS. 3A-3C: BRD4 Binding to CtIP Promoter and Enhancer and Effect on CtIP Transcription. (A) qRT-PCR analysis of cMYC and RBBP8 (the gene that encodes CtIP) in cells treated with 200 nM JQ1 (upper), or 200 nM AZD5153 (middle) for 24 hr, or after silencing of BRD4 or CtIP by siRNA for 48 hr (lower). (B) Schematic diagram of BRD4 binding regions in CtIP promoter and enhancer in ENCODE. Primers for ChIP-qPCR validation are indicated (upper). ChIP sequencing of anti-BRD4 at the RBBP8 locus in HCC1395 or T47D cells treated with JQ1 in GSE63581 dataset (lower). (C) HOC1 treated with vehicle or 200 nM JQ1 for 24 hr and subjected to ChIP with normal rabbit IgG, BRD4, H3K27Ac, H3K4Me1, or Pol-II antibody as indicated. ChIP samples were analyzed by qPCR using primers indicated in (B). Data across panels represent mean±SEM of three independent experiments. Student's t test: *p<0.05, ***p<0.001.

FIGS. 4A-4K: Effect of Downregulation of CtIP on DNA End Resection, Generation of ssDNA, and HR Function. (A) Representative images of BrdU and gH2AX staining under non-denaturing conditions at 4 hr after 10 Gy IR in HOC1 cells cultured with or without 200 nM JQ1. BrdU-positive cells were quantified below. Scale bar, 20 mm (B) Representative images of RPA foci in HOC1 cells after 24 hr BRD4 inhibition (200 nM JQ1 or siRNA) or CtIP downregulation (siRNA), and then treated with BMN673 (200 nM) for 48 hr. RPA foci-positive cells were quantified below. Scale bar, 20 mm (C) Western blotting of indicated proteins in HOC1 cells 24 hr after transfection with control, CtIP, or BRD4 siRNA, and then treated with 200 nM BMN673 for 48 hr. (D) Western blotting of indicated proteins in HOC1 cells treated with BMN673 (200 nM), JQ1 (200 nM), GSK1324726A (200 nM), or the indicated combination for 48 hr. (E) Western blot of indicated proteins in chromatin-bound fractions from HOC1 cells treated with BMN673 (200 nM), JQ1 (200 nM), or a combination for 48 hr. Histone H3 was used as marker for the chromatin-bound fraction. (F) Representative images of RAD51 and gH2AX foci in HOC1 cells after 24 hr BRD4 inhibition (200 nM JQ1 or siRNA) or CtIP downregulation (siRNA), and then treated with BMN673 (200 nM) for 48 hr. Scale bar, 5 mm (G) Comet assay in HOC1 cells treated with BMN673 (200 nM), JQ1 (200 nM), or a combination for 48 hr. DNA damage quantified via the percentage DNA in tails. Each data point represents at least 50 cells counted. Scale bar, 10 mm (H) Comet assay in HOC1 cells 24 hr after transfection with control, CtIP, or BRD4 siRNA, and then treated with 200 nM BMN673 for 48 hr. Each data point represents at least 50 cells counted. Scale bar, 10 mm (I) Twenty-four hours after transfection with control or CtIP siRNA, U2OS DR-GFP cells were transfected with the I-Sce1 endonuclease for 48 hr. HR efficiency of CtIP siRNA-treated cells was compared with control siRNA based on the percentage of GFP+ cells detected by flow cytometry. (J) Twenty-four hours after transfection with control, BRD4, or CtIP siRNA, clonogenic assay was performed with indicated dose of BMN673 for 7 days. Representative pictures are shown. (K) Cells were transfected with CtIP siRNA (50 nM) or treated with 200 nM JQ1, with or without 50 nM RAD51 siRNA, for 24 hr. Western blots of indicated proteins are in left panel. Cells were then treated for 96 hr with indicated doses of BMN673 and viability assessed (right). Short, short time exposure; long, long time exposure.

FIGS. 5A-5E: Effect of CtIP Expression on BRD4 Inhibition Induced DNA End Resection and HRDs. (A) Representative images (upper) and quantification (lower) of native BrdU foci staining in Dox-inducible GFP-CtIP or GFP-CtIP (T847A) HOC1 cells at 4 hr after 10 Gy IR plus 200 nM JQ1 treatment with or without Dox induction. Scale bar, 20 mm (B) Representative images (left) and quantification of positive cells (right) of RPA (upper) and RAD51 foci (lower) in Dox-inducible GFP-CtIP or GFP-CtIP (T847A) HOC1 cells treated with combination of 200 nM BMN673 and 200 nM JQ1 for 48 hr with or without Dox induction. Scale bar, 20 mm (C) Western blotting of indicated proteins in Dox-inducible GFP-CtIP and GFP-CtIP (T847A) HOC1 cells treated with BMN673 (200 nM), JQ1 (200 nM), or combination for 48 hr with or without Dox induction. (D) Representative pictures of clonogenic assay in Dox-inducible GFP-CtIP and GFP-CtIP (T847A) cells treated with BMN673 (200 nM), JQ1 (200 nM), or a combination for 10 days with or without Dox induction. (E) Relative colony formation rates of cell in (D) are presented as percent relative to DMSO. Data across studies represent mean±SEM of three independent experiments, Student's t test: *p<0.05, **p<0.01, ***p<0.001.

FIGS. 6A-6D: Effect of PARPi and BRD4i on Survival of Different Cell Lineages. (A) Dose-response curves of BMN673 or JQ1 alone or combined in 55 cancer cell lines treated with varying concentrations of the JQ1 and BMN673 for 96 hr. Combination index (CI) was calculated using CalcuSyn software with the Chou-Talalay equation. (B) BMN673 median inhibitory concentration (IC50) of (top) and selected mutations (middle) in cell lines. Red indicates a mutation in the respective gene, white indicates no mutation; red text indicates significant differences in frequency of mutations between PARPi-sensitive and -resistant cells (Pearson's chi-square test: p<0.05). The plot (bottom) shows the CtIP protein level in PARPi-sensitive and -resistant cells (Student's t test: p<0.001). (C) CI values of (top) and selected mutations (middle) in cell lines. Red indicates a mutation within the respective gene, white indicates no mutation; red text indicates significant differences in frequency of mutation between cells with or without synergism between BRD4i and PARPi (Pearson's chi-square test: p<0.05). The plot (bottom) shows the CtIP protein level in cells with or without synergism between BRD4i and PARPi (Student's t test: p=0.001). (D) Dose-response curves for BMN673 or JQ1 alone or combined for 96 hr in four normal human or murine proliferating cell lines. Data across panels represent mean±SEM of three independent experiments.

FIGS. 7A-7G: Effect of BRD4i on Acquired PARPi Resistance. (A) Dose-response curves of parental or PARPi-resistant OAW42 and A2780CP cells treated with BMN673 or JQ1 alone and combined for 96 hr. (B) Dose-response curves of parental or six individual monoclonal populations of PARPi-resistant OC316 treated with BMN673 (upper left) for 96 hr. Remaining graphs show dose-response curves of six individual monoclonal populations of PARPi-resistant OC316 treated with various concentrations of BMN673 alone or combined with 200 nM JQ1 for 96 hr. (C) Dose-response curves of UWB1.289 and UWB1.289-BRCA1 treated with BMN673 or JQ1 alone or combined for 96 hr. (D) Western blot of BRCA1/53BP1 knockdown efficiency in MCF10A stable lines or of 53BP1 knockdown efficiency in UWB1.289 and COV326 cell lines by siRNA for 48 hr. (E) Dose-response curves of BMN673 in parental, shBRCA1, or shBRCA1/53BP1 stable MCF10A cells with or without 200 nM JQ1 for 96 hr. (F) Dose-response curves of BMN673 in COV326 cells transfected with control or 53BP1 siRNA with or without 200 nM JQ1 for 96 hr. (G) Representative images of clonogenic assay in parental, shBRCA1, or shBRCA1/53BP1 stable MCF10A cells in the presence of the indicated inhibitor for 10 days. (H) Twenty-four hours after transfection with control or 53BP1 siRNA in UWB1.289 or COV326 cells, clonogenic assays were performed with indicated dose of BMN673 for 10 days. Representative pictures are shown. (I) Western blot of PARP1 in A2780CP cells after PARP1 silencing by siRNA for 48 hr (left). Dose-response curves in control or PARP1 knockdown cells treated with BMN673 or JQ1 alone or combined for 96 hr (right). (J) Western blot of PARP1 in parental or shPARP1 stable MDA-MB-231 cells (left). Dose-response curves in parental or shPARP1 stable MDA-MB-231 cells treated with BMN673 or JQ1 alone or combined for 96 hr (right). Short, short time exposure; long, long time exposure. Data represent mean±SEM of three independent experiments.

FIGS. 8A-8G: Efficacy of BRD4i and PARPi In Vivo. (A and B) Tumor volume curves (upper) or waterfall plot of tumor burden changes (lower) of OVCAR8 xenografts (A) and WU-BC3 PDX (B) mice treated with vehicle (0.5% hydroxypropylmethylcellulose and 0.2% Tween 80), BMN673 (0.333 mg/kg, oral gavage, per day), JQ1 (40 mg/kg, intraperitoneally, per day), or a combination of BMN673 and JQ1. (C-E) Tumor volume curves (upper) or waterfall plot of tumor burden changes (lower) of OVCAR3 (C) or PATX53 (D) xenografts or LPA1-T127 allograft (E) mice treated with vehicle (0.5% hydroxypropylmethylcellulose and 0.2% Tween 80), Olaparib (100 mg/kg, oral gavage, per day), AZD5153 (2.5 mg/kg, oral gavage, per day), or a combination of Olaparib and AZD5153. (F and G) Representative images of IHC with indicated antibodies in tumor tissues from OVCAR8 xenografts (F) or WU-BC3 PDX (G). Scale bar, 50 mm Data represent mean±SEM. ANOVA was used to compare differences among multiple groups. **p<0.01, ***p<0.001.

FIGS. 9A-9F: Effects of BRD4 inhibition on HR signature. (A) Heat maps of candidate genes after BRD4 inhibition using the GSE29799 dataset. (B) Heat map of unsupervised clustering of HRD gene signatures (upper), candidate genes (middle), and HRD scores (lower) after treatment with 500 nM JQ1 for 24 hr in different cell lines using the GSE66048 dataset. (C) Heat map of unsupervised clustering of HRD gene signatures (upper left), candidate genes (lower left), and HRD scores (right) after treatment with JQ1 at indicated dose or length in MM1S cells using the GSE44929 dataset. (D) Heat map of unsupervised clustering of HRD gene signatures (upper), candidate genes (middle), and HRD scores (lower) after treatment with 200 nM AZD5153 for 24 hr in different cell lines using the GSE85840 dataset. (E) Heat map of unsupervised clustering of HRD gene signatures (upper), candidate genes (middle), and HRD scores (lower) after treatment with 500 nM JQ1 for 24 hr in different cell lines using the GSE31365 dataset. (F) Heat map of unsupervised clustering of HRD gene signatures (upper), candidate genes (middle), and HRD scores (lower) after treatment with 1 μM JQ1 for 24 hr in Be2C cells using the GSE43392 dataset. Data of HRD scores after BRD4 inhibition represent mean±SEM for all panels. Statistical significances were determined using Student's t-test.

FIGS. 10A-10G: Effect of BRD4i on CtIP expression. (A) CtIP, RAD50, RAD51, and MRE11 proteins expression changes (BRD4i/DMSO) after treatment with BRD4i in each cell line (n=5) from RPPA in FIG. 2A. The top and bottom of the boxes indicate the 75th and 25th percentiles, respectively; line within the boxes indicates the median; lines above and below the boxes indicate the 95th and 5th percentiles, respectively. Outliers are indicated as dots. p values were calculated with Student's t test. (B) Western blot of CtIP, MRE11, RAD50, and NBS1 after treatment with 200 nM JQ1 for 48 hr in HeyA8, OVCAR8, and HOC7 ovarian cancer cells. (C) Western blot of indicated proteins in HeyA8 cells treated with indicated doses of JQ1 for 48 hr (left), or treated with 500 nM JQ1 for the indicated length (right). (D) Western blot of CtIP, RAD51, and BRCA1 after treatment with 200 nM JQ1 for 48 hr in a cell line panel. (E) Western blot of indicated proteins in HeyA8 and OVCAR8 cells after BRD4 silencing with siRNA for 48 hr. (F) Western blot of indicated proteins in HOC1 and HOC7 cells treated with indicated doses of AZD5153 for 48 hr. (G) Cell cycle assessment by flow cytometry (left) and quantification of percentage of cells in G₀/G₁, S, and G₂/M phase (right) in HOC1 and HOC7 cells treated with 200 nM JQ1 for 24 hr. Data represent the mean±SEM from three independent experiments.

FIGS. 11A-11K: Effects of BRD4 inhibition on DNA end resection, generation of ssDNA, and HR function. (A) Correlation between BRD4 protein expression and HRD scores in NCI60 and CCLE dataset. (B) Correlation between RBBP8 mRNA expression and HRD scores in NCI60 and CCLE dataset. (C) Representative images of BrdU and γH2AX staining under non-denaturing conditions at 4 hr after 10 Gy IR in HeyA8 cells cultured with or without 500 nM JQ1 (see STAR methods). BrdU positive cells are quantified on right. Scale bar, 20 μm. (D) Representative images of RPA foci staining at 4 hr after 10 Gy IR in HOC1 cells with or without 200 nM JQ1. Scale bar, 20 μm. (E) Western blot of indicated proteins in HeyA8 cells 24 hr after transfection with control, CtIP or BRD4 siRNA and then treated with 200 nM BMN673 for 48 hr. (F) Western blot of indicated proteins in HeyA8 cells treated with BMN673 (200 nM), JQ1 (500 nM), GSK1324726A (500 nM) or the indicated combination for 48 hr. (G) Western blot of indicated proteins in chromatin-bound fractions from HeyA8 cells treated with BMN673 (200 nM), JQ1 (500 nM), or combination for 48 hr. Histone H3 was used as a marker for chromatin-bound fraction. (H) Representative images of RAD51 and γH2AX foci in HeyA8 cells after BRD4 inhibition (500 nM JQ1 or siRNA BRD4) for 24 hr or CtIP downregulation (siRNA CtIP) for 24 hr and then treated with 200 nM BMN673 for 48 hr. Scale bar, 20 μm. (I) Representative images of RAD51 and γH2AX foci in HOC1 cells treated with BMN673 (200 nM), AZD5153 (200 nM), or combination for 48 hr. Scale bar, 20 μm. (J) Representative images of RAD51 and γH2AX foci in HOC1 cells 4 hr after 10 Gy IR with or without 200 nM JQ1. Scale bar, 20 μm. (K) Cells were transfected with indicated concentrations of CtIP siRNA or treated with indicated doses of JQ1 with or without 50 nM RAD51 siRNA for 24 hr. The dose of JQ1 and CtIP siRNA were pre-titrated to obtain similar levels of CtIP decrease. Western blots of indicated proteins are in left panel. Cells were then treated for 96 hr with indicated doses of BMN673 and viability assessed (right). Data across panels represent mean±SEM of three independent experiments. Statistical significances were determined using Student's t-test. **p<0.01.

FIGS. 12A-12D: Effects of RAD51 and BRCA1 on synergism between BRD4i and PARPi. (A) Western blot of indicated proteins in Dox inducible GFP-CtIP HOC1 cells treated with BMN673 (200 nM), AZD5153 (200 nM), or combination for 48 hr with or without Dox induction. (B) Western blot of RAD51, BRCA1 and CtIP in parental HOC1 and SKOC3 cells or cells ectopically expressing RAD51 or BRCA1 are shown (left). Representative pictures (middle) and quantification (right) of clonogenic assay in parental or cells ectopically expressing RAD51 or BRCA1 treated with BMN673 (100 nM), AZD5153 (200 nM), or combination for 10 days. Colony formation rates are presented as percentage relative to DMSO. (C) 24 hr after transfection with control, or CtIP siRNA in parental HOC1 or cells ectopically expressing RAD51 or BRCA1, clonogenic assay was performed in absence or presence of 100 nM BMN673 for 7 days. Representative pictures and quantification (lower right) of clonogenic assay are shown. Colony formation rates are presented as percentage relative to DMSO. (D) Cells stably expressing RAD51 or BRCA1 were established in Dox inducible GFP-CtIP HOC1 cells. Representative pictures (left) and quantification (right) of clonogenic assays in parental GFP-CtIP HOC1 cells or cells ectopically expressing RAD51 or BRCA1 treated with BMN673 (200 nM), AZD5153 (200 nM), or combination for 7 days with or without Dox induction. Colony formation rates are presented as percentage relative to DMSO. Data across panels represent mean±SEM of three independent experiments. Statistical significances were determined using Student's t-test. ***p<0.001.

FIGS. 13A-13H: Effects of inhibition of different BET Bromodomain proteins on synergism between BRD4i and PARPi. (A) Representative pictures of clonogenic assay in OVCAR8, HOC1, and HOC7 cells treated with the indicated concentrations of BMN673, JQ1, or combinations for 7 days. (B) 6 melanoma cell lines with NRAS mutation, 6 melanoma cell lines with BRAF mutation, 10 pancreatic cancer cell lines with KRAS mutation, 1 lung cancer cell line with KRAS mutation, and 1 colon cancer cell line with KRAS mutation were treated with varying concentrations of BMN673 or JQ1 alone or combined for 96 hr. Dose response curves are shown. CI was calculated using CalcuSyn software with the Chou-Talalay equation. (C) Primary breast cancer cell line (WU-BC3) with or without P53 knockdown were treated with varying concentrations of BMN673 or JQ1 alone or combined for 96 hr. Dose response curves are shown. (D) Dose response curves of BMN673 or BRD4i (GSK1324726A or AZD5153) alone or combined for 96 hr in four normal human or murine proliferating cell lines. (E) Dose response curves of BMN673 or BRD4i (AZD5153, GSK1324726A, or GSK1210151A) alone or combined for 96 hr in five ovarian cancer cell lines. (F) Western blot of indicated proteins in cells transfected with control, BRD2, BRD3, or BRD4 siRNA for 48 hr. (G) qRT-PCR analysis of RBBP8 in cells transfected with control, BRD2, BRD3, or BRD4 siRNA for 48 hr. (H) 24 hr after transfection with control, BRD2, BRD3, BRD4, or CtIP siRNA, clonogenic assay was performed in absence or presence of the indicated concentrations of BMN673, Olaparib or ABT888 for 7 days. Representative pictures (left) and quantification (right) of clonogenic assay are shown. Colony formation rates are presented as percentage relative to DMSO. Data across panels represent mean±SEM of three independent experiments. Statistical significances were determined using Student's t-test. **p<0.01, ***p<0.001.

FIGS. 14A-14H: Association of RBBP8, RAD51 and BRCA1 mRNA levels with synergism between BRD4i and PARPi, and association of BRD4 and CtIP with KRAS mutations. (A) Box plot of RBBP8, RAD51 or BRCA1 mRNA levels between cells with or without synergism between BRD4i and PARPi (left) or between PARPi sensitive and resistant cells (right). The top and bottom of the boxes indicate the 75th and 25th percentiles, respectively; line within the boxes indicates the median; lines above and below the boxes indicate the 95th and 5th percentiles, respectively. Outliers are indicated as dots. p values were calculated with Student's t test. (B) Western blot of CtIP, RAD51, and BRCA1 after treatment with 500 nM JQ1 for 48 hr in a panel of cells resistant to PARPi and BRD4i combinations. (C) Western blot of CtIP, RAD51, and BRCA1 after treatment with indicated concentrations of AZD5153 for 48 hr in EFE184 cells. (D) Western blot of indicated proteins after treatment with BMN673 (200 nM), JQ1 (200 nM), or combinations for 48 hr in EFE184 cells. (E) EFE184 cells were treated with vehicle or 200 nM JQ1 for 24 hr and subjected to ChIP assay with normal rabbit IgG, BRD4, H3K27Ac, H3K4Me1 or Pol-II antibodies. ChIP samples were analyzed by qPCR using primers targeting the regions indicated in FIG. 3B. Data represent mean±SEM of three independent experiments. (F) Western blot of indicated proteins in HPDE-iKRAS^{G12D} cell line with or without Dox induction for 24 hr. (G) HPDE-iKRAS^{G12D} cells were injected into athymic nude mice subcutaneously. Seven days after tumor injection, mice were treated with vehicle, Trametinib (2 mg/kg, oral gavage, per day) for 10 days with “Dox on” [via Dox diet (200 mg/kg; BioServ)], or “Dox off”. Tumor tissues from HPDE-iKRAS^{G12D} xenografts were subjected to IHC and probed with indicated antibodies. Representative IHC images are shown with treatment indicated. Scale bar, 100 μm. (H) Western blot of indicated proteins after treatment with 500 nM AZD6244 for 48 hr in four KRAS mutant cell lines.

FIGS. 15A-15E: Effect of inhibition of PARP enzyme activity on synergy with BRD4 inhibition. (A) Representative pictures (upper) and quantification (lower) of clonogenic assay in parental and PARP1 stable shRNA knockdown MDA-MB-231 cells treated with the indicated concentrations of BMN673, JQ1, or combination for 7 days. Colony formation rates are presented as percentage relative to DMSO. (B) 24 hr after transfection with control, or PARP1 siRNA, cells were treated with varying concentrations of JQ1 or PARPi (BMN673 or ABT888) alone or combined for 96 hr. (C) Dose response curves (left) and Western blots of PARP1 in cells transfected with control or PARP1 siRNA for 24 hr (right) are shown. Short: short time exposure, long: long time exposure. (D) Dose response curves of PARPi (Olaparib, or ABT888) or JQ1 alone or combined for 96 hr in five ovarian cancer cell lines. CI was calculated using CalcuSyn software with the Chou-Talalay equation. (E) Dose response curves of JQ1 alone or combined with BMN673 (500 nM), Olaparib (2 μM), or ABT888 (5 μM) for 96 hr in HOC1 cells. Dose response curves of BMN673 or JQ1 alone or combined for 96 hr in DT40 WT or DT40 PAPR1^{-/-} cells. Data represent mean±SEM of three independent experiments. Statistical significances were determined using Student's t-test. ***p<0.001.

FIGS. 16A-16F: Toxicity of PARPi, BRD4i and combination therapy in vivo. (A) LPA1-T127 tumor tissues were transplanted into the mammary fat pads of FVB mice. Eight days later, mice were randomized into treatment cohorts: vehicle (0.5% hydroxypropylmethylcellulose and 0.2% Tween 80), BMN673 (0.333 mg/kg, oral gavage, per day), JQ1 (40 mg/kg, I.P., per day), or combination of BMN673 and JQ1 (n=6 for each group). Tumor volume curves (left) and body weight curves of mice (right) are shown. Analysis of variance (ANOVA) was used for statistical significance. ***p<0.001. (B) Body weight curves of mice with OVCAR8 xenografts treated with vehicle (0.5% hydroxypropylmethylcellulose and 0.2% Tween 80), BMN673 (0.333 mg/kg, oral gavage, per day), JQ1 (40 mg/kg, I.P., per day), or combination of BMN673 and JQ1. (C) Body weight curves of mice with WU-BC3 PDX treated with vehicle (0.5% hydroxypropylmethylcellulose and 0.2% Tween 80), BMN673 (0.333 mg/kg, oral gavage, per day), JQ1 (40 mg/kg, I.P., per day), or combination of BMN673 and JQ1. (D) Body weight curves of mice with OVCAR3 xenograft treated with vehicle (0.5% hydroxypropylmethylcellulose and 0.2% Tween 80), Olaparib (100 mg/kg, oral gavage, per day), AZD5153 (2.5 mg/kg, oral gavage, per day), or combination of Olaparib and AZD5153. (E) Body weight curves of mice with LPA1-T127 allograft treated with vehicle (0.5% hydroxypropylmethylcellulose and 0.2% Tween 80), Olaparib (100 mg/kg, oral gavage, per day), AZD5153 (2.5 mg/kg, oral gavage, per day), or combination of Olaparib and AZD5153. (F) Plot of Albumin, ALT, AST, BUN, White blood cell (WBC), Red blood cell (RBC), Platelet count, and Hemoglobin levels in mice with LPA1-T127 allograft treated with vehicle, Olaparib (100 mg/kg, oral gavage, per day), AZD5153 (2.5 mg/kg, oral gavage, per day), or combination of Olaparib and AZD5153 (n=6 mice in each group). Statistical significances were determined using Student's t-test. *p<0.05. Data are presented as mean±SEM across all panels.

### DESCRIPTION OF ILLUSTRATIVE EMBODIMENTS

Poly (ADP-ribose) polymerase inhibitors (PARPi) are selectively active in cells with homologous recombination (HR) deficiency (HRD) caused by mutations in BRCA1, BRCA2, and other pathway members. The present studies sought small molecules that induce HRD in HR-competent cells to induce synthetic lethality with PARPi and extend the utility of PARPi. It was demonstrated that inhibition of bromodomain containing 4 (BRD4) induced HRD and sensitized cells across multiple tumor lineages to PARPi regardless of BRCA1/2, TP53, RAS, or BRAF mutation status through depletion of the DNA double-stand break resection protein CtIP (C-terminal binding protein interacting protein). Importantly, BRD4 inhibitor (BRD4i) treatment reversed multiple mechanisms of resistance to PARPi. Furthermore, PARPi and BRD4i were synergistic in multiple in vivo models. Therefore, the combination of BRD4 and PARP inhibitors has the potential to reverse or prevent the emergence of PARPi resistance and to increase the spectrum of patients who may benefit from the antitumor activity of PARP inhibitors.

Accordingly, in certain embodiments, the present disclosure provides compositions and methods for the treatment of cancer by a combination treatment of a PARP inhibitor and a BRD4 inhibitor. The present disclosure further provides methods for the prevention or reversal of PARP inhibitor resistance. In addition, the expression of CtIP in a subject can identify whether a patient is sensitive to PARP inhibition. For example, a subject with low CtIP expression may have a PARP sensitive cancer and a patient with high CtIP expression may have a PARP resistant cancer.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” refers to the stated value plus or minus 5%.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein, a composition that is “substantially free” of a specified substance or material contains 30%, 20%, 15%, more preferably 10%, even more preferably 5%, or most preferably 1% of the substance or material.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

### II. METHODS OF TREATMENT

In some embodiments, provided herein are methods for treating cancer in a subject comprising administering to the subject a therapeutically effective amount of a PARP inhibitor and a BRD4 inhibitor.

Exemplary solid tumors can include, but are not limited to, a tumor of an organ selected from the group consisting of pancreas, colon, cecum, stomach, brain, liver, gallbladder, head, neck, ovary, kidney, larynx, sarcoma, lung, bladder, uterus, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

BRD4 bromodomains can be selectively targeted with small-molecule inhibitors, such as JQ1, GSK1210151A (I-BET151), GSK1324726A (I-BET-726) and AZD5153, which compete with acetyl-lysine recognition to displace BRD4 from chromatin. The BRD4 inhibitor may be administered at a dose of from about 1 mg/day to about 100 mg/day. In some embodiments, the BRD4 inhibitor is administered once or twice daily at a dose of from about 10 mg to about 40 mg. In some embodiments, the BRD4 inhibitor is administered at doses of about 1 mg/kg per day, about 2 mg/kg per day, about 5 mg/kg per day, about 10 mg/kg per day, about 15 mg/kg per day, about 20 mg/kg per day, about 25 mg/kg per day, about 30 mg/kg per day, about 35 mg/kg per day, about 40 mg/kg per day, about 45 mg/kg per day, or about 50 mg/kg per day. The BRD4 inhibitor may be administered orally at a dose of 10 mg, 20 mg, or 40 mg tablets or capsules.

In some embodiments, the PARP inhibitor is administered at a dose of from about 20 mg/day to about 800 mg/day. In some embodiments, the PARP inhibitor is administered once or twice daily at a dose of from about 20 mg to about 400 mg. In some embodiments, the PARP inhibitor is administered at doses of about 1 mg/kg per day, about 2 mg/kg per day, about 5 mg/kg per day, about 10 mg/kg per day, about 15 mg/kg per day, about 20 mg/kg per day, about 25 mg/kg per day, about 30 mg/kg per day, about 35 mg/kg per day, about 40 mg/kg per day, about 45 mg/kg per day, about 50 mg/kg per day, about 60 mg/kg per day, about 70 mg/kg per day, about 80 mg/kg per day, about 90 mg/kg per day, about 100 mg/kg per day, about 125 mg/kg per day, about 150 mg/kg per day, about 175 mg/kg per day, about 200 mg/kg per day, about 250 mg/kg per day, or about 300 mg/kg per day. The PARP inhibitor may be administered in doses of 50, 100, or 150 oral tablets or capsules, such as at a daily dose of 300 or 400 mg/day. In some embodiments the PARP inhibitor is selected from the group consisting of talazoparib, niraparib, olaparib, veliparib, rucaparib, CEP 9722, talazoparib and BGB-290.

In certain embodiments, the BRD4 inhibitor and the PARP inhibitor are administered orally, intravenously, intraperitoneally, directly by injection to a tumor, topically, or a combination thereof. In some embodiments, the BRD4 inhibitor and the PARP inhibitor are administered as a combination formulation. In certain embodiments, the BRD4 inhibitor and the PARP inhibitor are administered as individual formulations. In some embodiments, the inhibitors are administered sequentially. In other embodiments, the inhibitors are administered simultaneously.

A. Combination Therapies

In certain embodiments, the methods provided herein further comprise a step of administering at least one additional therapeutic agent to the subject. All additional therapeutic agents disclosed herein will be administered to a subject according to good clinical practice for each specific composition or therapy, taking into account any potential toxicity, likely side effects, and any other relevant factors.

In certain embodiments, the additional therapy may be immunotherapy, radiation therapy, surgery (e.g., surgical resection of a tumor), chemotherapy, bone marrow transplantation, or a combination of the foregoing. The additional therapy may be targeted therapy. In certain embodiments, the additional therapy is administered before the primary treatment (i.e., as adjuvant therapy). In certain embodiments, the additional therapy is administered after the primary treatment (i.e., as neoadjuvant therapy).

A PARP inhibitor and BRD4 inhibitor may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below PARP inhibitor and/or BRD4 inhibitor therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PS Kpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs include ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, erb b2 and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin.

B. Pharmaceutical Compositions

In another aspect, provided herein are pharmaceutical compositions and formulations comprising a PARP inhibitor, BRD4 inhibitor and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of aqueous solutions, such as normal saline (e.g., 0.9%) and human serum albumin (e.g., 10%). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e g Zinc-protein complexes); and/or non-ionic surfactants such as polyethylene glycol (PEG).

### III. ARTICLES OF MANUFACTURE OR KITS

An article of manufacture or a kit is provided comprising a PARP inhibitor and BRD4 inhibitor is also provided herein. The article of manufacture or kit can further comprise a package insert comprising instructions for using the inhibitors to treat or delay progression of cancer in an individual. Any of the PARP and/or BRD4 inhibitors described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Combination of PARP and BRD4 Inhibition

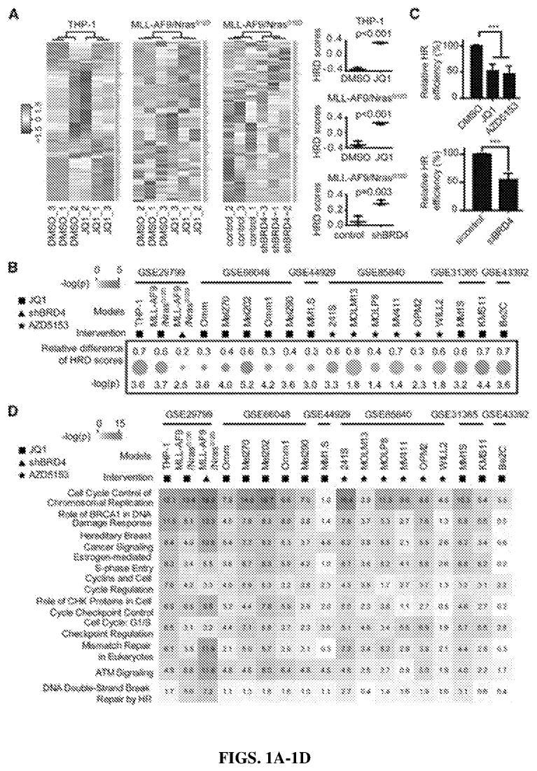

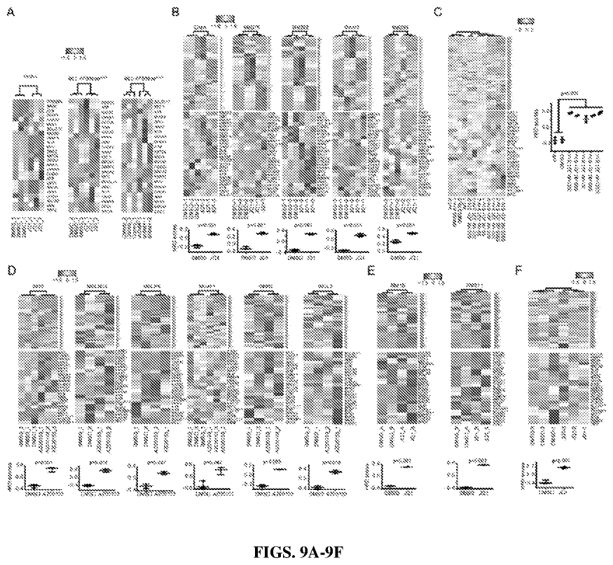

BRD4 Inhibition Induces an HRD Signature: The HR defect (HRD) gene signature (Peng et al., 2014) was applied to publicly available transcriptional profiling data with or without BRD4 inhibition to determine whether BRD4 inhibition impaired HR. BRD4i (JQ1) and BRD4 small hairpin RNA (shRNA) significantly elevated HRD scores in human THP-1 cells and in murine MLL-AF9/NrasG12D acute myeloid leukemia cells (Zuber et al., 2011) (FIG. 1A). Moreover, different BRD4i (JQ1, AZD5153) or BRD4 shRNA increased HRD score in human or murine tumors (FIGS. 1A, 1B, and 9A-9F). Strikingly, using a U2OS DR-GFP HR reporter assay, BRD4 inhibition with JQ1, AZD5153, or small interfering RNA (siRNA) attenuated HR repair (FIG. 1C). Therefore, BRD4 inhibition markedly decreases HR competence.

Ingenuity Pathway Analysis revealed that BRD4 inhibition altered expression of genes involved in DNA replication, BRCA1 in DNA damage response, hereditary breast cancer signaling, DNA damage checkpoint, cell cycle, and DNA repair pathway (FIG. 1D). These data further support BRD4 as a regulator of HR.

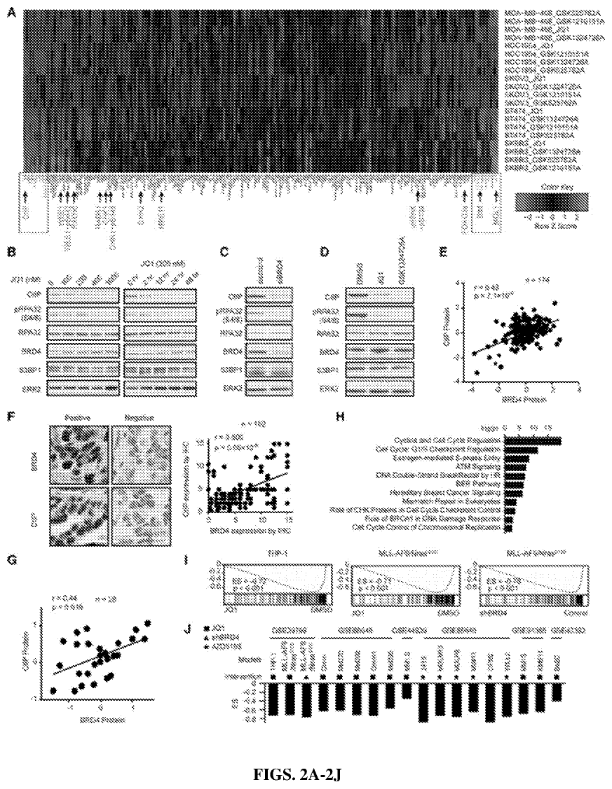

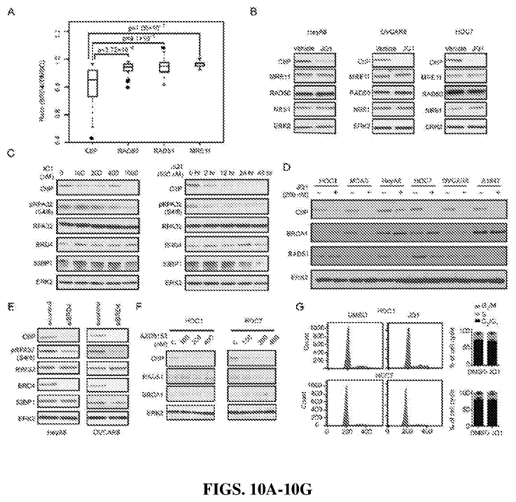

BRD4 Inhibition Decreases CtIP Expression: To identify mechanisms underlying the effect of BRD4 inhibition on HR, reverse phase protein arrays (RPPA) was used to assess signaling pathway perturbations in response to a clinical candidate (GSK525762A) and three experimental (GSK1210151A, GSK1324726A, and JQ1) BRD4i in five cancer cell lines. Replicates for each treatment condition (2D, spheroid 3D, and two time points [24 and 48 hr]) were averaged for each line (FIG. 2A). BRD4i markedly and consistently decreased CtIP, part of the MRN complex that commits cells to DSB repair. BRD4i extensively rewired protein networks, including multiple components of the DNA damage response pathway (WEE1, WEE1-pS642, RAD51, RAD50, CHK1, CHK1-pS345, CHK2, and MRE11) and induced DNA damage (gH2AX-pS139). In addition, BRD4i dysregulated the apoptosis pathway (BIM, FOXO3a, and MCL1). However, in contrast to CtIP, which was consistently downregulated under all conditions, the effects of BRD4i on RAD50, RAD51, and MRE11 were modest and variable (FIG. 10A). Thus, CtIP was focused on as a likely mediator of BRD4i effects.

CtIP is required for MRE11 to mediate DNA end resection, with loss of CtIP markedly decreasing DNA DSB repair through HR (Sartori et al., 2007; Yun and Hiom, 2009). Notably, JQ1 decreased CtIP and phosphorylated RPA32 (pRPA32 (S4/8)) protein in a dose- and time-dependent manner (FIGS. 2B, S2B, and 10C). In contrast, JQ1 did not markedly alter expression of other MRN complex proteins (FIG. 10B). BRD4 inhibition has recently been reported to downregulate RAD51 and BRCA1 mRNA (Yang et al., 2017). Thus, the effect of BRD4i on CtIP, RAD51, and BRCA1 protein levels was assessed. JQ1 modestly decreased RAD51 levels in HeyA8 and HOC7, and BRCA1 in MCAS, while it consistently decreased CtIP protein in all lines assessed (FIG. 10A). This is in general agreement with the RPPA data (FIGS. 2A and 10A), with the RPPA data appearing more sensitive than western blotting to subtle changes. To exclude potential off-target effects of JQ1, siRNA was used to knockdown BRD4. As expected, BRD4 siRNA also decreased CtIP and pRPA32 (S4/8) (FIGS. 2C and 10E). Similar results were also obtained with GSK1324726A (FIG. 2D) and AZD5153 (FIG. 10F). Moreover, BRD4i-induced CtIP decreases were not due to cell-cycle arrest (FIG. 10G). Correlation analysis of 174 cancer cell lines (MCLP) showed that BRD4 was positively correlated with CtIP expression (FIG. 2E). The positive correlation was verified in 102 ovarian cancer patient samples with immunohistochemical staining (IHC) (FIG. 2F). Consistent with these results, there was a significant, positive correlation between BRD4 protein and RBBP8 mRNA (encode CtIP protein) in NCI60 (FIG. 2G) and TCGA pan-cancer data (r=0.274, p=2.87 3 10-119). Using the Cancer Cell Line Encyclopedia (CCLE), a CtIP coexpression signature was derived that demonstrated high concordance with the BRD4 signature in terms of involved pathways (FIG. 2H) and was regulated by BRD4 inhibition (FIGS. 21 and 2J). Therefore, BRD4 appears to be a key regulator of CtIP protein level and function.

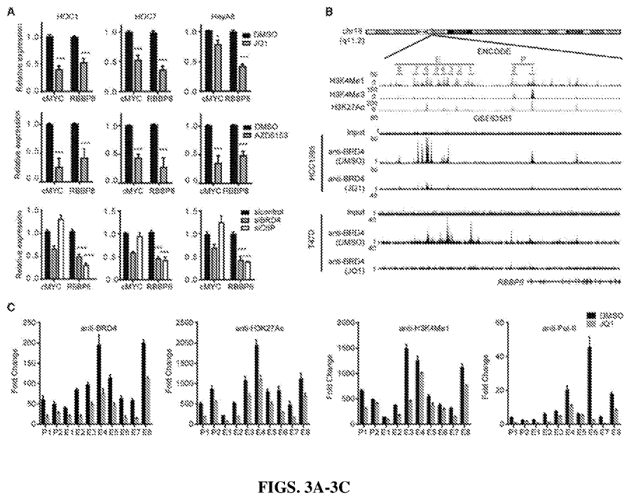

BRD4 Binds CtIP Promoter and Enhancers, Regulating CtIP Transcription: Transcription profiling demonstrated that RBBP8 is decreased by BRD4 inhibition (FIGS. 1A and 9). In support of this observation, RBBP8, along with cMYC, a key target of BRD4, were decreased by BRD4 inhibition (FIG. 3A). Thus, BRD4 inhibition likely alter CtIP levels through transcriptional effects. BRD4 regulates gene transcription by binding to enhancers and promoters of target genes (Love′ n et al., 2013; Yang et al., 2005). The ENCODE database and chromatin immunoprecipitation (ChIP) sequencing in GSE63581 (Shu et al., 2016) revealed BRD4 enrichment at the CtIP promoter and enhancer, with BRD4 enrichment decreased by BRD4i (FIG. 3B). Consistent with genome-wide studies, ChIP-qPCR of BRD4, H3K27Ac, H3K4Me1, and Pol-II antibodies, with primers located at CtIP promoter (P1 and P2) and enhancer (E1-E8) in HOC1, demonstrated BRD4 association with the CtIP promoter and enhancer, which was decreased with JQ1 treatment (FIG. 3C). Notably, JQ1 treatment also reduced H3K27Ac, H3K4Me1 at the CtIP promoter and enhancer. Further JQ1-mediated suppression of CtIP correlated with decreased association of RNA Pol-II with the CtIP promoter and enhancer, with Pol-II recently being reported to regulate gene transcription by binding to both promoters and enhancers (De Santa et al., 2010; Kim et al., 2010). Together, these data support the contention that CtIP is a direct target of BRD4, which is subject to JQ1-mediated repression at the transcriptional level.

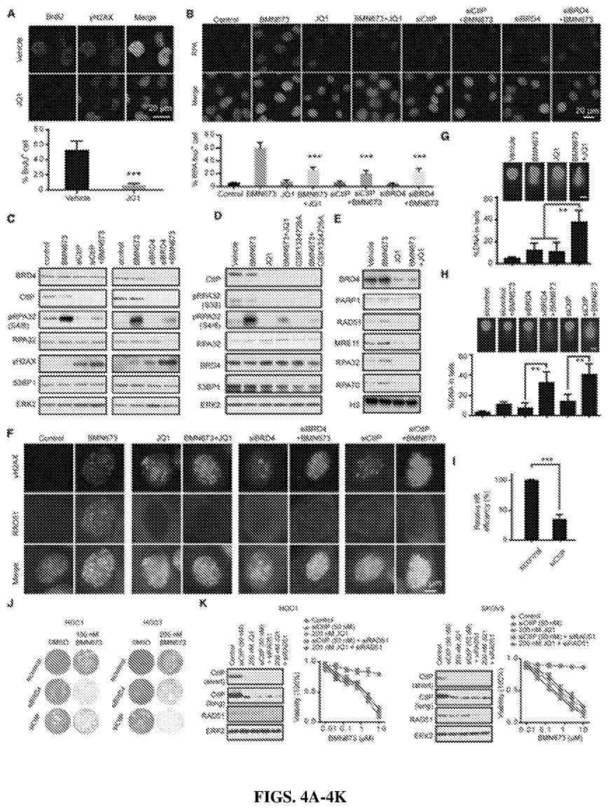

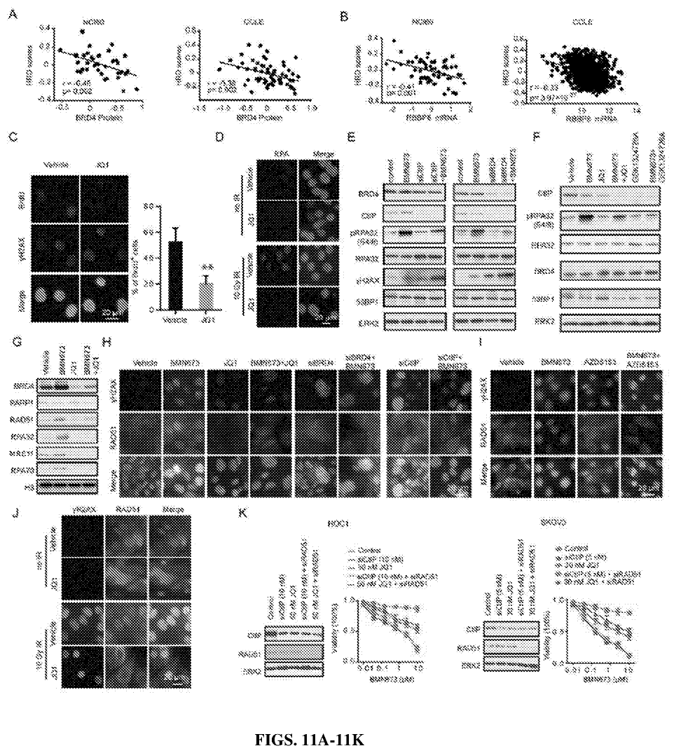

Downregulation of CtIP Is Sufficient to Impair DNA End Resection, Generation of ssDNA, and HR Function: CtIP is essential for efficient DNA end processing during DSB repair, with cells depleted for CtIP showing a defect in generation of ssDNA and subsequent formation of RPA foci (Polato et al., 2014; Yun and Hiom, 2009). It was thus hypothesized that BRD4 inhibition would block DNA end resection and HR through down-regulation of CtIP. Indeed, BRD4 protein and RBBP8 are negatively correlated with HRD score in both NCI60 and CCLE (FIG. 11A-B). To determine if BRD4 inhibition blocks ssDNA generation, cells were labeled with 5-bromodeoxyuridine (BrdU), and then immunofluorescence microscopy was employed using a BrdU antibody under non-denaturing conditions to detect stretches of ssDNA. JQ1 significantly reduced formation of ssDNA 4 hr after 10 Gy irradiation (IR) (FIGS. 4A and 11C), consistent with impaired resection. JQ1 also severely impaired RPA focus formation in response to PARPi (FIG. 4B) or IR (FIG. 11D). Consistent with CtIP being sufficient to explain the effects of BRD4 inhibition, both BRD4 and CtIP knockdown markedly impaired PARPi induced RPA focus formation (FIG. 4B). pRPA32 (S4/8) represents a surrogate marker for ssDNA that is generated by DNA end resection (Yun and Hiom, 2009). BRD4 inhibition decreased CtIP expression and strongly impaired PARPi-induced pRPA32 (S4/8) (FIG. 4C, 4D, 11E-F). Subcellular fractionation showed that BRD4i blocked recruitment of key DNA damage proteins to damaged chromosomes, including RAD51, RPA32, RPA70, and MRE11 (FIGS. 4E and 11G). Taken together, these results indicate that BRD4 inhibition attenuates efficient DSB resection, thereby impairing the subsequent formation of ssDNA.

RAD51 loading onto DNA requires ssDNA created by the CtIP/MRN complex. Compared with vehicle, JQ1 and AZD5153 retained RAD51 in the cytosol and decreased RAD51 nuclear foci after PARPi (FIG. 4F, 11H-I) or IR (FIG. 11J). Consistent with CtIP contributing to the effects of BRD4 inhibition, both siCtIP and siBRD4 inhibited PARPi-induced RAD51 foci formation (FIGS. 4F and 11H).

A comet assay was used to directly examine whether BRD4i would increase PARPi-induced DNA damage. Whereas JQ1 or BMN673 monotherapy modestly induced DNA damage, the combination increased accumulation of damaged DNA (FIG. 4G). Once again, knock down of BRD4 or CtIP was sufficient to recapitulate the effects of BRD4i (FIG. 4H).

DNA resection is the key commitment step for DSB repair by HR (Ira et al., 2004). These results suggested that BRD4 inhibition leading to loss of CtIP would decrease HR competency. Indeed, similar to BRD4i (FIG. 1C), both CtIP (FIG. 4I) and BRD4 (FIG. 1C) downregulation significantly decreased HR efficiency.

PARPi were developed to capitalize on synthetic lethality with HRD (Bryant et al., 2005; Farmer et al., 2005). Since BRD4 inhibition induced HRD, at least in part, through loss of CtIP, it was reasoned that knock down of BRD4 or CtIP would sensitize cells to PARPi. Indeed, knock down of BRD4 or CtIP markedly sensitized cells to PARPi (FIG. 4J). Importantly, at optimal doses, downregulation of CtIP with siRNA or BRD4i were indistinguishable in their effects on sensitization to PARPi. Further, RAD51 levels were not substantively altered by either CtIP downregulation or BRD4i, and concurrent knock down of RAD51 did not alter the response curve to PARPi (FIG. 4K). When lower doses of CtIP siRNA and JQ1 were used that suboptimally decrease CtIP levels, concurrent RAD51 knockdown induced a similar dose response shift for both CtIP siRNA and JQ1 (FIG. 11K). Thus, while CtIP downregulation was sufficient to mimic effects of BRD4i, when CtIP was partially downregulated, RAD51 knock down did alter PARPi sensitivity.

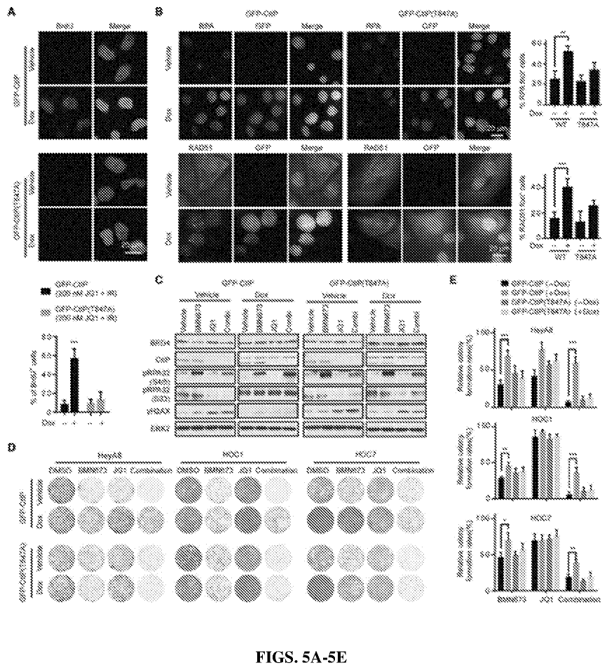

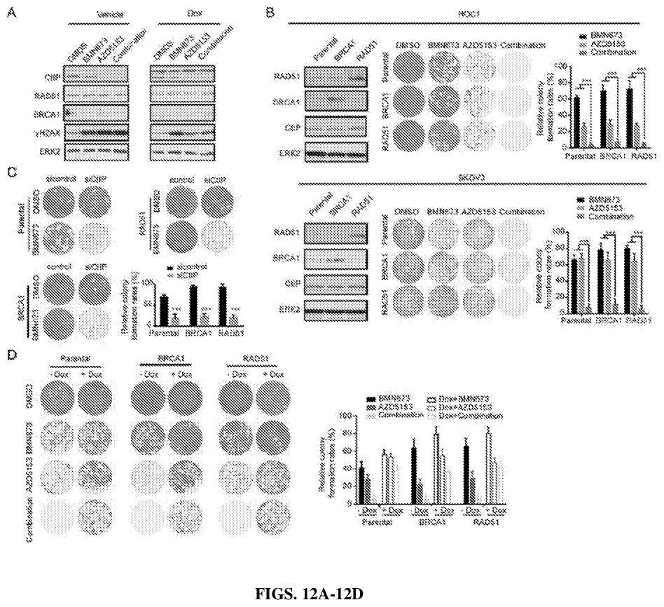

CtIP, but Not RAD51 or BRCA1, Partially Rescues BRD4 Inhibition Induced Defects in DNA End Resection and HR: To evaluate whether suppression of CtIP is necessary for BRD4 inhibition-induced defects in DNA end resection and HR function, Dox-inducible stable cell lines were generated expressing wild-type (WT) CtIP or inactive CtIP (T847A). CDK-mediated phosphorylation of CtIP on T847 was required for optimal CtIP function, thus conversion of threonine 847 to alanine (T847A) creates an inactive CtIP that is compromised for CtIP catalytic, ssDNA-, and RPA-binding activities (Huertas and Jackson, 2009; Polato et al., 2014). Ectopic expression of WT, but not inactive, CtIP increased ssDNA formation 4 hr after 10 Gy IR in the presence of JQ1 (FIG. 5A). Furthermore, expression of WT, but not inactive, CtIP partially restored PARPi-induced RPA and RAD51 foci formation (FIG. 5B) and pRPA32 (S4/8) (FIG. 5C) in the presence of JQ1. Ectopic expression of WT CtIP reduced DNA damage (gH2AX) caused by BRD4i and combination of BRD4i and PARPi (FIGS. 5C and 12A). Consistent with CtIP decrease being required for the effects of BRD4 inhibition, ectopic expression of WT, but not inactive, CtIP reversed, at least in part, the synergistic effects of PARPi and BRD4i (FIGS. 5D and 5E). Collectively, the data indicate that the catalytic, ssDNA- and RPA-binding activities of CtIP partially rescue impaired DNA end resection, RPA and RAD51 loading, and sensitization to PARPi induced by BRD4 inhibition.

Ectopic expression of BRCA1 or RAD51 did not rescue cells from the effects of combination treatment (FIG. 12B). Furthermore, knock down of CtIP sensitized cells to PARPi even when BRCA1 and RAD51 were overexpressed (FIG. 12C). In addition, induced CtIP expression rescued the effects of combination treatment, while ectopic expression of BRCA1 and RAD51 alone had no effect (FIG. 12D). Thus, decreases in CtIP, but not RAD51 and BRCA1, appear to be necessary and sufficient for synergistic effects of BRD4i and PARPi in the model systems assessed.

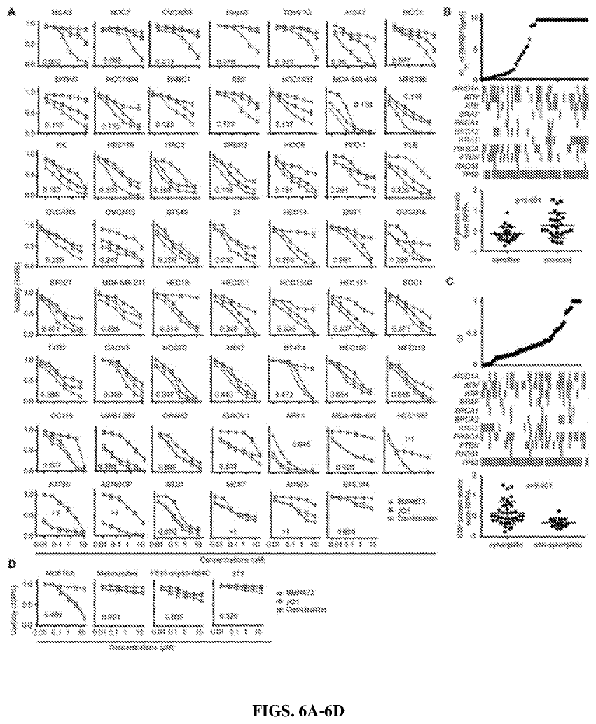

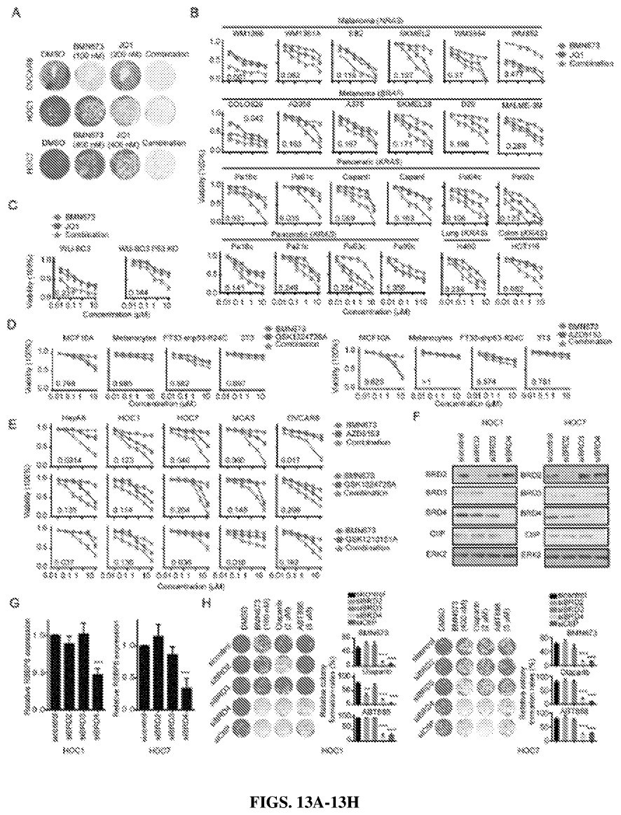

PARPi and BRD4i Demonstrate Synergy in Multiple Cancer Lineages: Based on the ability of BRD4 inhibition to compromise HR, the effects of combination treatment with PARPi and BRD4i was assessed. Of 55 cancer cell lines tested, 40 lines demonstrated synergy as assessed by the CalcuSyn model (combination index <0.5, FIG. 6A). The majority of the lines (9/15) that failed to demonstrate synergy were highly sensitive to BMN673 (OAW42, A2780CP, A2780, UWB1.289, and OC316, ARK1, HCC1187, BT20, MDA-MB-436) (FIG. 6A). Further, combinations at low concentrations induced significant decreases in clonogenicity (FIG. 13A) compared with treatment with either inhibitor alone.

As demonstrated previously (Sun et al., 2017), KRAS mutation is a potent inducer of PARPi resistance (FIG. 6B). Strikingly, synergism of PARPi and BRD4i was most clearly manifest in KRAS mutant cells (FIG. 6C). This may, in part, be due to resistance of KRAS mutant cell lines to PARPi alone, making synergistic activity more readily manifest. The synergistic activity of the combination was independent of ARID1A, ATM, ATR, BRCA1/2 PIK3CA, PTEN, and TP53 status, consistent with generalizability and independence from intrinsic HRD status. The striking synergistic effects of PARPi and BRD4i in KRAS mutant cells led us to test additional RAS/BRAF mutant cells across multiple lineages. Strikingly, the combination was synergistic in 12 NRAS or BRAF mutant melanoma cells, as well as 11 of 12 KRAS mutant pancreatic, lung, or colon cancer cells (except Pa09c cells) (FIG. 13B). The combination was also synergistic in the parental WU-BC3 patient-derived xenografts (PDX) and in a P53 knockdown clone (Ma et al., 2012), which was resistant to PARPi (FIG. 13C). In contrast, the combination was not synergistic in non-tumorigenic MCF10A (breast epithelial cells), melanocytes, FT33-shp53-R24C (immortalized human fallopian tube secretory epithelial cells), and 3T3 (mouse embryonic fibroblasts) (FIGS. 6D and 13D).

In addition to JQ1, three other BRD4i demonstrated similar patterns of synergy with PARPi (FIG. 13E). Combined with synergy observed between BRD4 or CtIP knockdown with PARPi (FIG. 4J), these results suggested the synergistic effects of BRD4i and PARPi were indeed due to specific BRD4 inhibition. To verify this hypothesis, BRD2, BRD3, and BRD4 were knocked down individually with siRNA. Only BRD4 depletion decreased CtIP protein and transcript levels (FIG. 13F-G). Furthermore, only BRD4 depletion sensitized cells to PARPi (FIG. 13H).

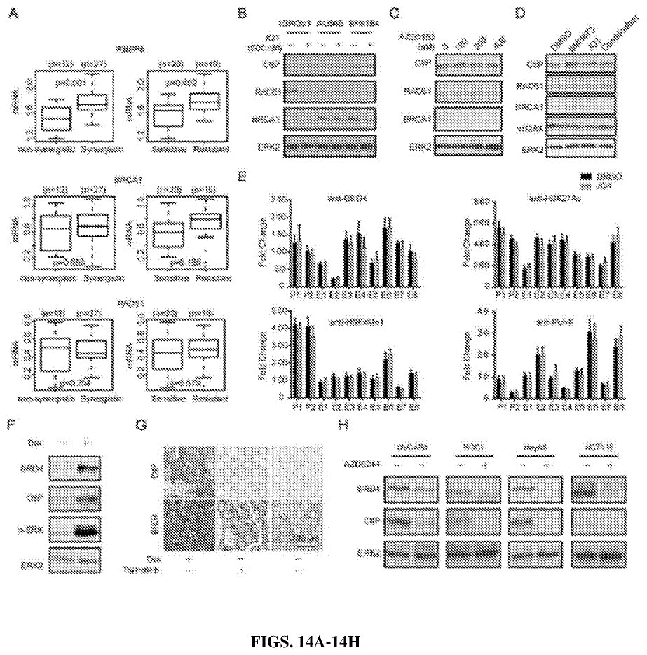

In 55 cell lines tested for response, CtIP expression was much lower in PARPi-sensitive cells, indicating that CtIP may serve as a marker of PARPi sensitivity (FIG. 6B). Moreover, higher CtIP protein and mRNA, but not RAD51 or BRCA1 mRNA, was a marker of synergism of PARPi and BRD4i (FIGS. 6C and 14A), consistent with the concept that CtIP depletion contributes to PARPi and BRD4i synergy.

Six of the lines tested were resistant to PARPi alone as well as to the combination. Strikingly, BRD4i failed to alter CtIP levels in the three resistant cell lines tested. In contrast, RAD51 was decreased in IGROV1, and BRCA1 decreased in EFE184 (FIG. 14B-C). Moreover, the combination did not increase DNA damage (gH2AX), consistent with the lack of synergism in EFE184 (FIG. 14D). ChIP-qPCR data showed that, although BRD4, H3K27Ac, H3K4Me1, and Pol-II bind to the promoter and enhancer of CtIP in EFE184 cells, BRD4i did not decrease binding activity (FIG. 14E). Together, these data further support the concept that downregulation of CtIP contributes to synergistic activity of PARPi and BRD4i.

It was further sought to identify mechanisms by which KRAS mutant cell lines would be selectively sensitive to PARPi and BRD4i combinations. Induction of activated KRASG12D in HPDE cells induced both BRD4 and CtIP (FIG. 14F), which was reversed by a selective MEKi in vivo (FIG. 14G). Consistent with this result, MEKi decreased both BRD4 and CtIP in multiple cancer cell lines with RAS/MAPK pathway activation or mutant KRAS (FIG. 14H). Together, increases in BRD4 and CtIP protein in response to RAS/MAPK pathway activation likely contribute to sensitivity of KRAS mutant cells to PARPi and BRD4i combinations.

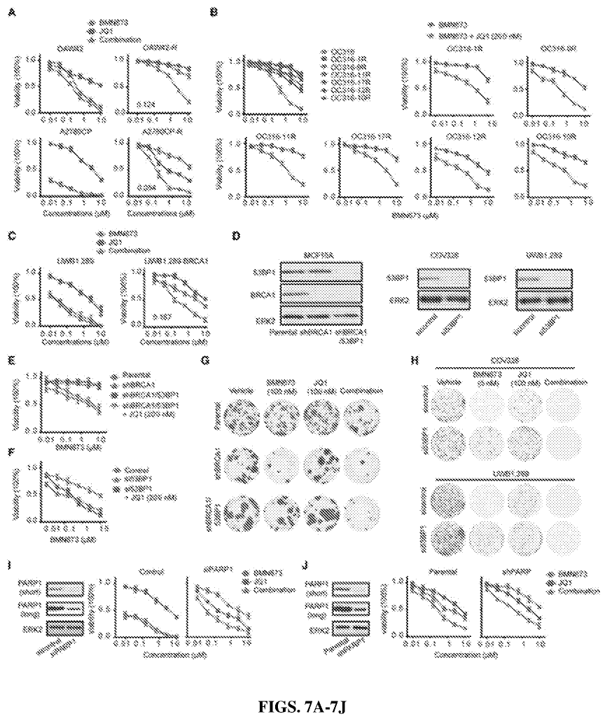

BRD4i Resensitizes Acquired PARPi Resistance: Although many patients benefit from PARPi, acquired PARPi resistance is an almost universal occurrence. To explore whether BRD4i could resensitize PARPi-resistant cells to PARPi, several PARPi-resistant models representing different mechanisms of PARPi resistance were used. First, PARPi-resistant cells were developed by culturing sensitive cells (A2780CP, OAW42, and OC316) in the continued presence of BMN673. It was demonstrated previously that A2780CP_R has acquired mutations in KRAS, as well as in MAP2K1 (Sun et al., 2017). JQ1 resensitized A2780CP_R, OAW42_R and OC316_R to PARPi (FIGS. 7A and 7B). Second, UWB1.289 is a BRCA1-mutant line (BRCA12594delC). UWB1.289-BRCA1, which stably expresses WT BRCA1, is resistant to PARPi and mimics BRCA1/2, RAD51C, or RAD51D reversion mutations. BRD4i and PARPi combinations were synergistic in UWB1.289-BRCA1, albeit with lower efficacy than PARPi in parental cells (FIG. 7C). Third, loss of 53BP1 normalizes HRDs, rescues the lethality of BRCA1 deficiency, and leads to PARPi resistance in BRCA1-null cells and animal models (Bunting et al., 2010). CtIP-dependent DNA end resection rescues genomic stability and HR function of BRCA1/53BP1-deficient cells (Bunting et al., 2010; Polato et al., 2014; Xu et al., 2015). Since BRD4i decreased CtIP expression, it was proposed that BRD4i would resensitize BRCA1/53BP1 double-deficient cells to PARPi. 53BP1 knockdown rendered BRCA1 knockdown MCF10A cells, as well as BRCA1 mutant UWB1.289 and COV362 cells, resistant to PARPi. In all cases, JQ1 reversed resistance mediated by 53BP1 knockdown (FIG. 7D-7H). Fourth, decreased PARP1 levels have been identified as a mechanism of PARPi resistance, particularly to the effects of “PARP trapping” inhibitors in model systems (Byers et al., 2012; Murai et al., 2012). Synergistic effects of PARPi and BRD4i were also observed in cells with knock down of PARP1 (FIG. 71, 7J, 15A-B).

Taken together, BRD4i resensitizes multiple mechanisms of acquired PARPi resistance that have been observed in patients and model systems to PARPi. Thus, BRD4i and PARPi combinations may prevent emergence of PARPi resistance, or may be effective in the emerging population of patients where PARPi are initially active and then fail.

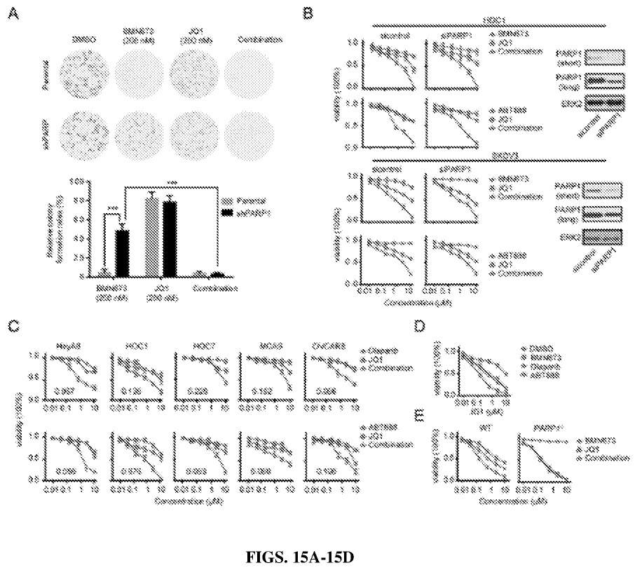

Inhibition of PARP Enzyme Activity Appears Sufficient for Synergy with BRD4i: The different PARPi currently available in the clinic effectively inhibit the enzyme activity of PARP, but vary in their ability to trap PARP on DNA. BMN673 is most active, Olaparib, Niraparib, and Rucaparib intermediate in activity, whereas ABT888/Veliparib has the weakest PARPi trapping activity (Murai et al., 2012). High levels of PARP1 are required for trapping activity of PARPi to be manifest and, thus, a role for trapping activity can be elucidated by testing activity of different PARPi, as well as by determining the effects of partial knock down of PARP1 (Murai et al., 2012). Indeed, as noted above, the synergistic effects of PARPi and BRD4i are not altered by partial knock down of PARP1 (note residual PARP1 remains) (FIG. 71, 7J, 15A-B). Furthermore, synergistic effects were maintained in combinations with PARPi of lower PARP trapping potential (Olaparib) and with minimal PARP trapping activity (ABT888) with similar combination indices indicative of synergy (FIG. 15C). In addition, 5 mM ABT888 (lower concentration than required for PARP trapping [50 mM; Murai et al., 2012]) synergized with JQ1, similar to Olaparib and BMN673 (FIG. 15D). Finally, subcellular fractionation did not demonstrate increased PARP1 trapping on DNA with combination treatment (FIGS. 4E and 11G). Together, inhibition of PARP enzyme activity appeared sufficient for the synergistic effects of BRD4i and PARPi, consistent with marked HRD induced by BRD4 inhibition. PARP1−/− DT40 cells completely lack PARP enzyme activity because avian cells lack PARP2. PARP1−/− DT40 are resistant to BMN673, consistent with the lack of PARP1/2. Cytotoxic effects of combinational treatment with BMN673 and JQ1 are indistinguishable from the effects of JQ1 alone in PARP1−/− DT40 cells. Importantly, the dose-response curve of BMN673 and JQ1 combinations in WT DT40 is equivalent to that of JQ1 in PARP1−/− DT40 (FIG. 15E). This was consistent with activity of combination being dependent on the presence of PARP and further argues that the effects of PARPi in combination with BRD4i are on-target.

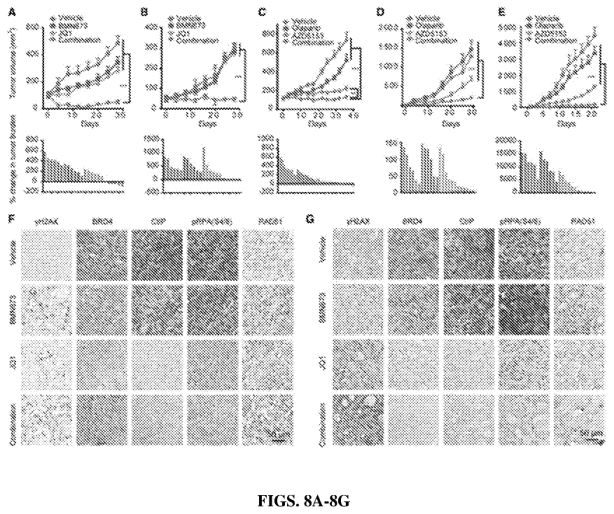

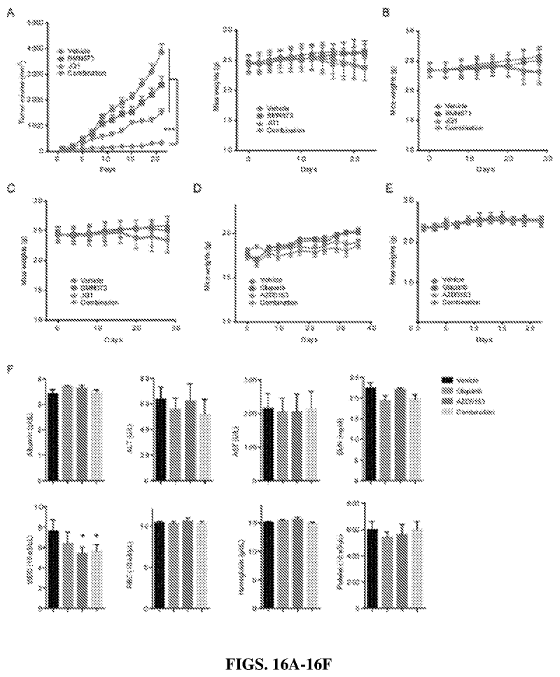

BRD4i and PARPi Are Synergistic In Vivo: On the basis of synergy of BRD4i and PARPi in vitro, BRD4i and PARPi combinations were explored in five different in vivo models. OVCAR8 is a KRASP121H mutant (the mutant is a variant of unknown significance, but the line has an activated RAS/MAPK pathway [Sun et al., 2017]) ovarian cancer line, OVCAR3 is a TP53 mutant, RAS WT ovarian cancer line, WU-BC3 is a breast cancer PDX (HER2-E subtype with WT TP53) (Ma et al., 2012), PATX53 is a KRASG12D and TP53 mutant pancreatic PDX, and LPA1-T127 is an MMTV-LPA receptor transgene-induced transplantable tumor that acquired a spontaneous KRASQ61H mutation (Federico et al., 2017). Similar to human PDX, the LPA1-T127 tumor has never been cultured on plastic and may thus be more representative of the heterogeneity of human breast cancers. Furthermore, LPA receptor transgene-induced tumors are late onset, heterogeneous, and are associated with an inflammatory response similar to human cancers (Liu et al., 2009). Strikingly, in OVCAR8, WU-BC3, and LPA1-127, the JQ1 and PARPi combination induced prolonged tumor control (FIGS. 8A, 8B, and 16A) with tumor regression in the OVCAR8 xenograft. The combination of JQ1 and BMN673 was well tolerated, with modest weight loss late in treatment that was not different from JQ1 alone (FIG. 16A-C). To demonstrate generalizability and since JQ1 is not a clinical candidate, the clinically approved PARPi (Olaparib) and a selective, orally available, and bivalent BRD4i (AZD5153) (Rhyasen et al., 2016) were assessed, which is entering clinical evaluation. In OVCAR3 ovarian cancer cells, as well as in KRAS mutant PATX53 and LPA1-T127, all of which are HR competent and resistant to PARPi alone, the combination markedly inhibited tumor growth to a much greater degree than either compound alone (FIG. 8C-8E). Indeed, in the OVCAR3 model, AZD5153 resulted in 83% tumor growth inhibitions (TGI), Olaparib showed minimal effect at 35% TGI and the combination treatment resulted in near stasis with 98% TGI. Moreover, only the combination treatment induced tumor regression (8/10 mice) (FIG. 8C). The Olaparib and AZD5153 combination was tolerated for the study duration (FIG. 16D-E). To further evaluate safety of the combination, toxicity analysis of Olaparib with AZD5153 was performed in the T127 model. The numbers of white blood cells in the AZD5153 and combination therapy group showed a slight decrease but remained in the normal range compared with the vehicle group. No changes in red blood cells, platelets, or hemoglobin were detected. Serum chemistry panels did not reveal changes in albumin, ALT, AST, and BUN levels (FIG. 16F).

IHC of OVCAR8 and WU-BC3 PDX tumors at study termination recapitulated the in vitro studies. JQ1 increased gH2AX, which was further increased by combination with BMN673. As expected, CtIP and its direct downstream effector pRPA32 (S4/8) were decreased in JQ1-treated tumors, which was not reversed by addition of BMN673 (FIGS. 8F and 8G).

It was demonstrated that decreased CtIP transcription appears to be a major contributor to the effects of BRD4 inhibition on HR function and to be necessary and sufficient for much of the synergy between PARPi and BRD4i. CtIP inhibition has previously been associated with PARPi sensitivity (Lin et al., 2014; Wang et al., 2016). Importantly, enforced expression of CtIP was sufficient to, at least in part, reverse the effects of BRD4i on DNA end resection, HR function, and PARPi sensitivity. DNA replication fork reversal and fork stability are emerging mechanisms of PARPi resistance independent of HR repair (Ray Chaudhuri et al., 2016). CtIP has also been demonstrated to induce replication fork recovery in a FANCD2-dependent manner (Yeo et al., 2014). The effects of CtIP on DNA repair as well as replication stress induced by tumorigenesis may contribute to DNA damage observed in cells treated with BRD4i herein. Thus, BRD4 inhibition induced CtIP loss may contribute to PARPi sensitivity through multiple CtIP-dependent mechanisms. However, as BRD4 regulates the expression of many molecules, there may be additional effects of BRD4i that contribute to sensitization to PARPi either independent of CtIP loss or in cooperation with CtIP loss.

### Example 2—Materials and Methods

Clinical Specimens: Use of ovarian cancer samples was approved by the Ethics or Institutional Review Board of Tongji Hospital, Tongji Medical College, Huazhong University of Science and Technology, China, in accordance with the Declaration of Helsinki. Informed consent was obtained from all subjects. 102 serous ovarian cancer stage IIIC or IV (International Federation of Gynecology and Obstetrics staging) samples were collected between January 2009 and October 2013. Samples were routinely fixed immediately after surgery in 10% formalin for approximately 24 hr at room temperature. After fixation, samples were dehydrated, incubated in xylene, infiltrated with paraffin, and finally embedded in paraffin.

WU-BC3 PDX, which was established in Washington University (Li et al., 2013), was obtained from Dr. Helen Piwnica-Worms in Department of Experimental Radiation Oncology in MDACC (MD Anderson Cancer Center) (Ma et al., 2012). PATX53 was obtained from Dr. Michael P. Kim in Department of Surgical Oncology in MDACC. WU-BC3 and PATX53 PDX were under IRB approved protocol by the ethics committee of the Washington University, or the MDACC respectively, with written informed consent for formation and use of PDX.

Animal Studies: 6 week old female NCRNU-F sp/sp mice were purchased from Taconic and were used for OVCAR8 xenografts, WU-BC3 PDX and PATX53 PDX experiments. 6 week old female FVB mice were purchased from Taconic and were used for LPA-T127 syngeneic breast cancer model experiments. Tumors were injected or transplanted into female mice of approximately 8-10 weeks of age. All mice were housed under pathogen-free conditions at MDACC AAALAC (Association for the Assessment and Accreditation of Laboratory Animal Care) accredited facility. All animal experiments with these models were conducted in compliance with the National Institute of Health guidelines for animal research and approved by the Institutional Animal Care and Use Committee of the MDACC.

6 weeks old female C.B-17 scid mice were purchased from Charles River Laboratories and used for OVCAR3 xenografts. Tumor cells were injected into female mice of approximately 8-10 weeks of age. All mice were housed under pathogen-free conditions at AstraZeneca AAALAC accredited facility. All animal experiments were conducted in compliance with the National Institute of Health guidelines for animal research and approved by the Institutional Animal Care and Use Committee of AstraZeneca.

Cell Lines: All human cell lines were authenticated by fingerprinting using short tandem repeat testing and were verified to be free of mycoplasma contamination. All cell lines were maintained in a 5% CO₂ incubator at 37° C. Detail information about cells are provided in Table 1. pCW-GFP-CtIP was a gift from Daniel Durocher (Addgene plasmid #71109) (Orthwein et al., 2015). pCW-GFP-CtIP (T847A) was generated by site-directed mutagenesis. Cells infected with viruses expressing these cDNAs were maintained in 2 mg/mL puromycin to generate stable cell lines. GFP-CtIP and GFP-CtIP (T847A) expression were induced with 100 nM doxycycline (Dox). HOC1, SKOV3, HOC1-GFP-CtIP stably expressing RAD51 and BRCA1 were established through standard procedural.

Generation of PARPi Resistant Cells: To generate PARPi resistant cells, A2780CP and OAW42 were subjected to gradual increases in BMN673 concentrations until cells grew in the presence of 10 mM of BMN673 (3-4 months from initial exposure).

For PARPi resistant OC316 clones, cells were subjected to gradual increases in BMN673 concentrations until cells grew in the presence of 5 mM of the BMN673 (3-4 months from initial exposure). Monoclonal cell populations of the OC316 resistant cells are isolated by limiting dilution. Individual clones demonstrated different degrees of resistance to PARPi.

Cells were cultured in the absence of BMN673 for a minimum of 1 month before they were used for experiments.

RPPA: Five breast and ovarian cancer cell lines, [BT474 (PIK3CA_Mut, HER2_Amp), HCC1954 (PIK3CA_Mut and HER2_Amp), MDA-MB-468 (EGFR_Overexpression and PTEN_Mut), SKBR3 (HER2_Amp), SKOV3 (PIK3CA_Mut and HER2_Amp)], were cultured in Matrigel (3D) or monolayer (2D) and treated for 24 hr or 48 hr, respectively, with DMSO or BRD4i (GSK1210151A, GSK1324726A, GSK525762A, and JQ1). Median inhibitory concentration (IC₅₀) was determined experimentally for JQ1 for each line for 2D and 3D conditions with other inhibitors being used at 2 concentrations (100 nM and 1000 nM). Protein lysates were analyzed by RPPA in MDACC CCSG (The Cancer Center Support Grant) supported RPPA Core. Antibodies and approaches are described at the RPPA website (https://www.mdanderson.org/research/research-resources/core-facilities/functional-proteomics-rppa-core.html). For visualization, 2D and 3D, concentrations and time were averaged for each cell line. Heat map represents “rank-ordered” changes induced by BRD4i treatment, calculated by summing median-centered protein amount normalized to DMSO. Western Blot: To prepare whole cell lysates, cells were lysed with RIPA buffer (Thermo Fisher Scientific) supplemented with Halt™ Protease and Phosphatase Inhibitor (EDTA-free) Cocktail (Thermo Fisher Scientific). After thorough mixing and incubation at 4° C. for 10 min, lysates were centrifuged at 15,000 g at 4° C. for 15 min, and supernatants were collected. To prepare subcellular fraction of nuclear soluble and chromatin-bound fraction, cells were treated with indicated drugs, and then cells were collected. For fractionation, a Subcellular Protein Fractionation kit (Thermo Fisher Scientific) was used following the manufacturer's instructions. The protein content of the cell was determined, and the cellular lysates were separated by 10% SDS-PAGE, and electro-transferred onto polyvinylidene difluoride (PVDF) membranes. After being blocked with 5% non-fat milk in TBST, the membranes were incubated with primary antibodies at 4° C. overnight, followed by 1:2000 horseradish peroxidase (HRP)-conjugated secondary antibody (Abcam) for 1 hr. Bands were visualized using a Pierce™ ECL Western Blotting Substrate (Thermo Fisher Scientific). Primary antibodies used are listed in Key Resources Table.

qRT-PCR: Total RNA was isolated using RNeasy Plus Mini Kit (Qiagen) according to the manufacturer's protocol. RNA was treated with RNase-free DNase set (Qiagen) to remove contaminating genomic DNA. cDNA was synthesized using High Capacity cDNA Reverse Transcription Kit (Applied Biosystems). qRT-PCR was performed using SYBR Green Master Mix (Applied Biosystems). Data were analyzed by the DDCT method using GAPDH as a housekeeping gene. The sequences of primers used are listed in Table 2.

Site-directed Mutagenesis: pCW-GFP-CtIP was a gift from Daniel Durocher (Addgene plasmid #71109) (Orthwein et al., 2015). Mutant pCW-GFP-CtIP (T847A) was generated by targeting WT pCW-GFP-CtIP using QuikChange II XL Site-Directed Mutagenesis kit (Agilent Technologies) with primers list in Table 2. Mutagenesis reactions were prepared in PCR tubes on ice: 5 mL of 10× reaction buffer, 2 mL pCW-GFP-CtIP plasmid DNA (10 ng), 1.25 mL of mutagenic primer (CtIP_T847A_F at 100 ng/mL), 1.25 mL of mutagenic primer (CtIP_T847A_R at 100 ng/mL), 1 mL of dNTP mix, 3 mL of QuickSolution reagent, 36.5 mL PCR-quality water to a final volume of 50 mL were mixed then 1.0 mL PfuUltra HF DNA polymerase (2.5 U/mL) was fused. Tubes were placed in the cycler to begin the PCR reaction for 18 cycles. 1 mL of the Dpn I restriction enzyme (10 U/ml) was added directly to amplification reaction and mixed thoroughly and incubated at 37° C. for 1 hour. Then 2 ml of the Dpn I-treated DNA was transformed to XL10-Gold Ultracompetent Cells. Mutation was confirmed by sequencing.

TABLE 1
Cell lines.

| Cell lines | Species | Gender | Culture Media | Source | Identifier |
|---|---|---|---|---|---|
| A1847 | Human | female | RPMI-1640 + 10% | MDACC characterized Cell | N/A |
| | | | FBS + Insulin | line Core | |
| A2780 | Human | female | RPMI-1640 + 5% | MDACC characterized Cell | N/A |
| | | | FBS | line Core | |
| A2780CP | Human | female | RPMI-1640 + 5% | MDACC characterized Cell | N/A |
| | | | FBS | line Core | |
| ARK1 | Human | female | RPMI-1640 + 10% | MDACC characterized Cell | N/A |
| | | | FBS | line Core | |
| ARK2 | Human | female | RPMI-1640 + 10% | MDACC characterized Cell | N/A |
| | | | FBS | line Core | |
| AU565 | Human | female | RPMI-1640 + 10% | MDACC characterized Cell | N/A |
| | | | FBS | line Core | |
| BT20 | Human | female | RPMI-1640 + 10% | MDACC characterized Cell | N/A |
| | | | FBS | line Core | |
| BT474 | Human | female | RPMI-1640 + 10% | MDACC characterized Cell | N/A |
| | | | FBS | line Core | |
| BT549 | Human | female | RPMI-1640 + 5% | MDACC characterized Cell | N/A |
| | | | FBS | line Core | |
| CAOV3 | Human | female | RPMI-1640 + 20% | MDACC characterized Cell | N/A |
| | | | FBS | line Core | |
| ECC1 | Human | female | RPMI-1640 + 10% | MDACC characterized Cell | N/A |
| | | | FBS | line Core | |
| EF027 | Human | female | RPMI-1640 + 10% | MDACC characterized Cell | N/A |
| | | | FBS | line Core | |
| EFE184 | Human | female | RPMI-1640 + 10% | MDACC characterized Cell | N/A |
| | | | FBS | line Core | |
| EI | Human | female | DMEM + 10% | MDACC characterized Cell | N/A |
| | | | FBS | line Core | |
| ENT1 | Human | female | RPMI-1640 + 10% | MDACC characterized Cell | N/A |
| | | | FBS | line Core | |
| ES2 | Human | female | McCoy's 5a + 10% | MDACC characterized Cell | N/A |
| | | | FBS | line Core | |
| HAC2 | Human | female | DMEM/F12 + 10% | MDACC characterized Cell | N/A |
| | | | FBS | line Core | |
| HCC1187 | Human | female | RPMI-1640 + 10% | MDACC characterized Cell | N/A |
| | | | FBS | line Core | |
| HCC1500 | Human | female | RPMI-1640 + 10% | MDACC characterized Cell | N/A |
| | | | FBS | line Core | |
| HCC1937 | Human | female | RPMI-1640 + 10% | MDACC characterized Cell | N/A |
| | | | FBS | line Core | |
| HCC1954 | Human | female | RPMI-1640 + 10% | MDACC characterized Cell | N/A |
| | | | FBS | line Core | |
| HCC70 | Human | female | RPMI-1640 + 10% | MDACC characterized Cell | N/A |
| | | | FBS | line Core | |
| HEC108 | Human | female | RPMI-1640 + 10% | MDACC characterized Cell | N/A |
| | | | FBS | line Core | |
| HEC116 | Human | female | DMEM + 10% FBS | MDACC characterized Cell | N/A |
| | | | | line Core | |
| HEC151 | Human | female | RPMI-1640 + 10% | MDACC characterized Cell | N/A |
| | | | FBS | line Core | |
| HEC1A | Human | female | McCoy's 5a + 10% | MDACC characterized Cell | N/A |
| | | | FBS | line Core | |
| HEC1B | Human | female | McCoy's 5a + 10% | MDACC characterized Cell | N/A |
| | | | FBS | line Core | |
| HEC251 | Human | female | RPMI-1640 + 10% | MDACC characterized Cell | N/A |
| | | | FBS | line Core | |
| HeyA8 | Human | female | RPMI-1640 + 10% | MDACC characterized Cell | N/A |
| | | | FBS | line Core | |
| HOC1 | Human | female | RPMI-1640 + 5% | MDACC characterized Cell | N/A |
| | | | FBS | line Core | |
| HOC7 | Human | female | RPMI-1640 + 5% | MDACC characterized Cell | N/A |
| | | | FBS | line Core | |
| HOC8 | Human | female | RPMI-1640 + 5% | MDACC characterized Cell | N/A |
| | | | FBS | line Core | |
| IGROV1 | Human | female | RPMI-1640 + 10% | MDACC characterized Cell | N/A |
| | | | FBS | line Core | |
| KK | Human | female | RPMI-1640 + 10% | MDACC characterized Cell | N/A |
| | | | FBS | line Core | |
| KLE | Human | female | RPMI-1640 + 10% | MDACC characterized Cell | N/A |
| | | | FBS | line Core | |
| MCAS | Human | female | RPMI-1640 + 10% | MDACC characterized Cell | N/A |
| | | | FBS | line Core | |
| MCF7 | Human | female | RPMI-1640 + 5% | MDACC characterized Cell | N/A |
| | | | FBS | line Core | |
| MCF10A | Human | female | Special Medium | MDACC characterized Cell | N/A |
| | | | (ATCC Webpage) | line Core | |
| MDA-MB- | Human | female | RPMI-1640 + 5% | MDACC characterized Cell | N/A |
| 231 | | | FBS | line Core | |
| MDA-MB- | Human | female | DMEM + 10% FBS | MDACC characterized Cell | N/A |
| 436 | | | | line Core | |
| MDA-MB- | Human | female | DMEM/F12 + 10% | MDACC characterized Cell | N/A |
| 468 | | | FBS | line Core | |
| MFE296 | Human | female | RPMI-1640 + 20% | MDACC characterized Cell | N/A |
| | | | FBS + Insulin | line Core | |
| MFE319 | Human | female | RPMI-1640 + 10% | MDACC characterized Cell | N/A |
| | | | FBS | line Core | |
| OAW42 | Human | female | RPMI-1640 + 5% | MDACC characterized Cell | N/A |
| | | | FBS | line Core | |
| OC316 | Human | female | RPMI-1640 + 5% | MDACC characterized Cell | N/A |
| | | | FBS | line Core | |
| OVCAR3 | Human | female | RPMI-1640 + 5% | MDACC characterized Cell | N/A |
| | | | FBS | line Core | |
| OVCAR4 | Human | female | RPMI-1640 + 5% | MDACC characterized Cell | N/A |
| | | | FBS | line Core | |
| OVCAR5 | Human | female | RPMI-1640 + 5% | MDACC characterized Cell | N/A |
| | | | FBS | line Core | |
| OVCAR8 | Human | female | RPMI-1640 + 10% | MDACC characterized Cell | N/A |
| | | | FBS | line Core | |
| PANC1 | Human | female | DMEM + 10% FBS | MDACC characterized Cell | N/A |
| | | | | line Core | |
| PEO-1 | Human | female | RPMI-1640 + 5% | MDACC characterized Cell | N/A |
| | | | FBS | line Core | |
| SKBR3 | Human | female | RPMI-1640 + 20% | MDACC characterized Cell | N/A |
| | | | FBS | line Core | |
| SKOV3 | Human | female | RPMI-1640 + 5% | MDACC characterized Cell | N/A |
| | | | FBS | line Core | |
| T47D | Human | female | RPMI-1640 + 5% | MDACC characterized Cell | N/A |
| | | | FBS | line Core | |
| TOV21G | Human | female | 1:1 Medium | MDACC characterized Cell | N/A |
| | | | 199/MCDB105 + | line Core | |
| | | | 15% FBS | | |
| UWB1.289 | Human | female | RPMI- | MDACC characterized Cell | N/A |
| | | | 1640:MEGM + 3% | line Core | |
| | | | FBS | | |
| UWB1.289- | Human | female | RPMI- | MDACC characterized Cell | N/A |
| BRCA1 | | | 1640:MEGM + 3% | line Core | |
| | | | FBS | | |
| Pa01C | Human | N/A | RPMI-1640 + 10% | Laboratory of Dr. Anirban | N/A |
| | | | FBS | Maitra | |
| Pa02C | Human | N/A | RPMI-1640 + 10% | Laboratory of Dr. Anirban | N/A |
| | | | FBS | Maitra | |
| Pa03C | Human | N/A | RPMI-1640 + 10% | Laboratory of Dr. Anirban | N/A |
| | | | FBS | Maitra | |
| Pa04C | Human | N/A | RPMI-1640 + 10% | Laboratory of Dr. Anirban | N/A |
| | | | FBS | Maitra | |
| Pa09C | Human | N/A | RPMI-1640 + 10% | Laboratory of Dr. Anirban | N/A |
| | | | FBS | Maitra | |
| Pa16C | Human | N/A | RPMI-1640 + 10% | Laboratory of Dr. Anirban | N/A |
| | | | FBS | Maitra | |
| Pa18C | Human | N/A | RPMI-1640 + 10% | Laboratory of Dr. Anirban | N/A |
| | | | FBS | Maitra | |
| Pa21C | Human | N/A | RPMI-1640 + 10% | Laboratory of Dr. Anirban | N/A |
| | | | FBS | Maitra | |
| CAPAN I | Human | male | RPMI-1640 + 10% | MDACC characterized Cell | N/A |
| | | | FBS | line Core | |
| CAPAN II | Human | male | RPMI-1640 + 10% | MDACC characterized Cell | N/A |
| | | | FBS | line Core | |
| WM1366 | Human | male | RPMI-1640 + 10% | MDACC characterized Cell | N/A |
| | | | FBS | line Core | |
| WM1361A | Human | N/A | RPMI-1640 + 10% | MDACC characterized Cell | N/A |
| | | | FBS | line Core | |
| SB2 | Human | N/A | RPMI-1640 + 10% | MDACC characterized Cell | N/A |
| | | | FBS | line Core | |
| SKMEL2 | Human | male | RPMI-1640 + 10% | MDACC characterized Cell | N/A |
| | | | FBS | line Core | |
| WM3854 | Human | male | DMEM + 10% | MDACC characterized Cell | N/A |
| | | | FBS | line Core | |
| WM852 | Human | male | DMEM + 10% | MDACC characterized Cell | N/A |
| | | | FBS | line Core | |
| COLO829 | Human | male | DMEM + 10% | MDACC characterized Cell | N/A |
| | | | FBS | line Core | |
| A2058 | Human | male | DMEM + 10% | MDACC characterized Cell | N/A |
| | | | FBS | line Core | |
| A375 | Human | female | DMEM + 10% | MDACC characterized Cell | N/A |
| | | | FBS | line Core | |
| SKMEL28 | Human | male | RPMI-1640 + 10% | MDACC characterized Cell | N/A |
| | | | FBS | line Core | |
| D29 | Human | N/A | RPMI-1640 + 10% | MDACC characterized Cell | N/A |
| | | | FBS | line Core | |
| MALME- | Human | male | RPMI-1640 + 10% | MDACC characterized Cell | N/A |
| 3M | | | FBS | line Core | |
| H460 | Human | male | DMEM + 10% | MDACC characterized Cell | N/A |
| | | | FBS | line Core | |
| HCT116 | Human | male | RPMI-1640 + 5% | MDACC characterized Cell | N/A |
| | | | FBS | line Core | |
| WU-BC3 | Human | female | DMEM/F12 + 10% | Laboratory of Dr. Helen | N/A |
| | | | FBS | Piwnica-Worms | |
| WU-BC3 | Human | female | DMEM/F12 + 10% | Laboratory of Dr. Helen | N/A |
| P53 KD | | | FBS | Piwnica-Worms | |
| MDA-MB- | Human | female | RPMI-1640 + 5% | Laboratory of Dr. Mien-Chic | N/A |
| 231 | | | FBS | Hung | |
| MDA-MB- | Human | female | RPMI-1640 + 5% | Laboratory of Dr. Mien-Chic | N/A |
| 231 PARP1 | | | FBS | Hung | |
| KD | | | | | |
| Melanocytes | Human | N/A | Special Medium | ATCC | PCS-200- |
| | | | (Look up on ATCC | | 013 |
| | | | Webpage) | | |
| FT33-shp53- | Human | female | WIT + 15% FBS | ABM | T0609 |
| R24C | | | | | |
| 3T3 | Mouse | N/A | DMEM + 10% FBS | MDACC characterized Cell | N/A |
| | | | | line Core | |
| DT40 | Chicken | N/A | RPMI-1640 + 10% | Laboratory of Dr. Shunichi | N/A |
| | | | FBS + 5% Chicken | Takeda | |
| | | | serum | | |
| DT40 | Chicken | N/A | RPMI-1640 + 10% | Laboratory of Dr. Shunichi | N/A |
| PARP1−/− | | | FBS + 5% Chicken | Takeda | |
| | | | serum | | |
| HPDE- | Human | N/A | KSFM | Laboratory of Dr. Kenneth | N/A |
| iKRASG12D | | | | L. Scott | |
| U2OS DR- | Human | female | McCoy's 5a + 10% | MDACC characterized Cell | N/A |
| GFP | | | FBS | line Core | |

TABLE 2
Oligonucleotides.

| | | | | | SEQ |
|---|---|---|---|---|---|
| | | | | | ID |
| Purpose | Name | Source | Identifier | Sequence | NO: |
| ChIP-qPCR | P1-Forward | Sigma | N/A | ATTGTCGTCGTGCCTCGAAT | 1 |
| | P1-Reverse | Sigma | N/A | AAACCCTTTCCACCTACCCG | 2 |
| | P2-Forward | Sigma | N/A | CACCCAGGCAAATGTTTGGTC | 3 |
| | P2-Reverse | Sigma | N/A | GCTTAGCCTTGAGGAGCGAG | 4 |
| | E1-Forward | Sigma | N/A | CTGTTGCTGAGCTACCAAGGA | 5 |
| | E1-Reverse | Sigma | N/A | TCATCAGGCAACCAAGCCAT | 6 |
| | E2-Forward | Sigma | N/A | GTGGCTCCCTACACCAAACA | 7 |
| | E2-Reverse | Sigma | N/A | GCCAGAAGCCCAGTGGTAAT | 8 |
| | E3-Forward | Sigma | N/A | ATTATGTCGCCGGAACTGGT | 9 |
| | E3-Reverse | Sigma | N/A | AGACCGAGGAAGACCTGACT | 10 |
| | E4-Forward | Sigma | N/A | TGGTTCCCCAGTTCTGTTGG | 11 |
| | E4-Reverse | Sigma | N/A | CCTGGACATGTCTGGAAAGTGA | 12 |
| | E5-Forward | Sigma | N/A | TTACCAGCTTACAGACTCCTGC | 13 |
| | E5-Reverse | Sigma | N/A | TGTGGGAGTTCCCTGAGTCTAA | 14 |
| | E6-Forward | Sigma | N/A | CATGCCAACACCCGCTCATA | 15 |
| | E6-Reverse | Sigma | N/A | CCCCAACGGGGTTGTCAAAA | 16 |
| | E7-Forward | Sigma | N/A | CACACAAGCCAGCTTTTACTGT | 17 |
| | E7-Reverse | Sigma | N/A | ACTTGGTAGGGGCACATTGG | 18 |
| | E8-Forward | Sigma | N/A | AAAATGTTCTCCCGCCAGCA | 19 |
| | E8-Reverse | Sigma | N/A | CAAGCATGCCCAGTGTTTGC | 20 |
| qRT-PCR | cMYC- | Sigma | N/A | CAGCGACTCTGAGGAGGAAC | 21 |
| | Forward | | | | |
| | cMYC- | Sigma | N/A | GCTGGTGCATTTTCGGTTGT | 22 |
| | Reverse | | | | |
| | RBBP8- | Sigma | N/A | GGCTTATGTGATCGCTGTGC | 23 |
| | Forward | | | | |
| | RBBP8- | Sigma | N/A | ATGTGCTTTGGCCATTGGAG | 24 |
| | Reverse | | | | |
| Site- | CtIP_T847A_F | Sigma | N/A | TTCCGCTACATTCCACCCAACG | 25 |
| directed | | | | CTCCAGAGAATTTTTGGGAAGT | |
| mutagenesis | | | | T | |
| | CtIP_T847A_R | Sigma | N/A | AACTTCCCAAAAATTCTCTGGA | 26 |
| | | | | GCGTTGGGTGGAATGTAGCGGA | |
| | | | | A | |
| siRNA | CtIP_siRNA | GE | L- | N/A | |
| | | Dharmacon | 011376- | | |
| | | | 00-0005 | | |
| | BRD4 siRNA | GE | L- | N/A | |
| | | Dharmacon | 004937- | | |
| | | | 00-0005 | | |
| | 53BP1 siRNA | GE | L- | N/A | |
| | | Dharmacon | 003548- | | |
| | | | 00-0005 | | |
| | PARP1 siRNA | GE | L- | N/A | |
| | | Dharmacon | 006656- | | |
| | | | 03-0005 | | |
| | BRD2 siRNA | GE | L- | N/A | |
| | | Dharmacon | 004935- | | |
| | | | 00-0005 | | |
| | BRD3 siRNA | GE | L- | N/A | |
| | | Dharmacon | 004936- | | |
| | | | 00-0005 | | |
| | ON- | GE | | N/A | |
| | TARGETplus | Dharmacon | | | |
| | Non-targeting | | D- | | |
| | Pool | | 001810- | | |
| | | | 10-05 | | |

RNA Interference: All siRNAs employed in this study were ON-TARGET plus siRNA SMARTpools purchased from GE Dharmacon (Table 2). RNA interference (RNAi) transfections were performed using Lipofectamine™ 3000 Transfection Reagent (Invitrogen) in a forward transfection mode using manufacturer's guidelines. Except when stated otherwise, siRNAs were transfected with the amounts of siRNA oligos at 40 nM final concentration.

CCLE and NCI60 Dataset Gene expression profiles (Gene transcript level z score) for correlations analysis in NCI60 human tumor cell lines were obtained using the web-based tool provided by CellMiner. Gene expression data for Cancer Cell Line Encyclopedia (CCLE) (CCLE_expression_CN_muts_GENEE_2010-04-16) were downloaded. The correlations between gene expressions were determined by Pearson's correlation test with R.

Microarray Analysis and IPA Analysis: Gene expression datasets of GSE29799 (Zuber et al., 2011), GSE66048 (Ambrosini et al., 2015), GSE44929 (Love′ n et al., 2013), GSE85840 (Rhyasen et al., 2016), GSE31365 (Delmore et al., 2011), and GSE43392 (Puissant et al., 2013) were downloaded from Gene Expression Omnibus (GEO) (https://www.ncbi.nlm.nih.gov/geo). Raw data were subjected to intensity normalization using affy package in R (Bioconductor), followed by log transformation and quantile normalization. Normalized data were checked for quality and determined to be free of outliers by analysis using box plots, density plots and MA plots. Differential expression genes after BRD4 inhibition were calculated using a linear model provided by the limma package in R based on the cutoffs: 2 for absolute fold change, 0.05 for p value. Then, results were imported into Ingenuity Pathway Analysis (IPA) and a core analysis feature was used to reveal dysregulated canonical pathway after BRD4 inhibition.

HRD Score Acquisition from HRD Signature HRD signature consisting of 230 differentially expressed genes was obtained as previously described (Peng et al., 2014). Normalized gene expression data (GSE29799, GSE66048, GSE44929, GSE85840, GSE31365, and GSE43392) after BRD4 inhibition were subjected to unsupervised clustering with these 230 genes. HRD scores were determined by calculating the Pearson's correlations between median centered gene expression levels for HRD signature and gene expression levels for a given sample (Peng et al., 2014).

ChIP-Seq Analysis: ChIP-seq data for T47D and HCC1935 cells from GSE63581 (Shu et al., 2016) were aligned versus hg19 human genome for mapping using bowtie. For peaking calling, MACS2 was used to get the bam files, which were converted to bigwig files later in deeptools and loaded in Intergrative Genomics Viewer (IGV) for final visualization and cross comparison. Specifically, ChIP-seq for T47D and HCC1935 cells treated with JQ1 and vehicle control were compared with input.

CtIP Co-expression Signature and GSEA Analysis: CtIP co-expression signature was constructed base on genes whose expressions are correlated with RBBP8 levels in the CCLE dataset at cBioPortal. 326 genes were selected using Pearson's correlation coefficient R 0.3 as cutoff. Then these 326 genes were imported into Ingenuity Pathway Analysis (IPA) for network and pathway analysis.

For GSEA analysis against CtIP co-expression signature, these 326 genes were incorporated into the GSEA Desktop v3.0 as the CtIP co-expression signature. Then normalized gene expression data (GSE29799, GSE66048, GSE44929, GSE85840, GSE31365, and GSE43392) were used to calculate enrichment of CtIP co-expression signature after BRD4 inhibition by Benjamin-Hochberg (B-H) method.

Viability Measurements: Five thousand cells were seeded into sterile 96-well plates and treated with indicated drug combinations for 96 hr. DMSO was used as a vehicle. PrestoBlue® Cell Viability Reagent (Thermo Fisher Scientific) was used to assess cell viability. Background values from empty wells were subtracted and data normalized to vehicle-treated control. Synergistic effects between both compounds were calculated using the Chou-Talalay equation in CalcuSyn software, which takes into account both potency (IC₅₀) and shape of the dose-effect curve. CI<0.5 indicates synergism, CI between 0.5 to 1 indicates additive effects, and CI>1 indicates antagonism.

Immunohistochemical Staining (IHC) Tissues were fixed in 10% formalin overnight and embedded in paraffin. 4 mm paraffin embedded sections were first deparaffinized in xylene. IHC were carried out with EnVision Detection Systems HRP. Rabbit/Mouse (DAB+) kit (Agilent) following manufacturer's instructions. Endogenous peroxidase was blocked by incubation with 0.3% hydrogen peroxide for 15 min. Antigen retrieval was performed by boiling the slides in citrate buffer (10 mM, pH 6.0) in a water bath for 20 mM. Slides were rinsed in PBS Tween 0.05% and blocked for 30 mM with 5% bovine serum albumin (BSA). Slide were incubated overnight at 4° C. with primary antibodies (anti-BRD4,

#13440S, 1:200; anti-CtIP, #9201S, 1:200 from Cell Signaling Technology; anti-RAD51, PC130, 1:100; anti-g-H2AX (Ser139), clone JBW301, 1:500 from Millipore Corp; and anti-pRPA32(S4/8), A300-245A, 1:1000 from Bethyl Laboratories), followed by 1 hr with Labelled Polymer-HRP at room temperature. Negative controls were treated identically, but without primary antibody. Subsequently, slides were incubated with DAB+ Chromogen. Slides were counterstained with hematoxylin. After mounting, slides were observed under microscope and photographed.

The IHC score for BRD4 and CtIP staining are the average of the score of tumor-cell staining multiplied by the score of staining intensity. Tumor cell staining was assigned a score using a semi-quantitative five-category grading system: 0, no tumor-cell staining; 1, 1-10% tumor-cell staining; 2, 11-25% tumor-cell staining; 3, 26-50% tumor-cell staining; 4, 51-75% tumor-cell staining; and 5, >75% tumor-cell staining Staining intensity was assigned a score using a semi-quantitative four-category grading system: 0, no staining; 1, weak staining; 2, moderate staining; and 3, strong staining Every core was assessed individually and the mean of three readings was calculated for every case. Tumor cell staining score was determined separately by two independent experts simultaneously under the same conditions. In rare cases, discordant scores were reevaluated and scored on the basis of consensus opinion.

Alkaline Single-Cell Agarose Gel Electrophoresis (Comet) Assays: Alkaline comet assays were performed with Comet Assay Kit (Trevigen) using manufacturer's instructions. Briefly, cell suspensions were embedded in LM (low melting) Agarose and deposited on comet slides. Slides were incubated for 1 hr at 4° C. in lysis solution, followed by immersing slides in freshly prepared alkaline unwinding solution (pH >13) for 20 min at room temperature in the dark. Electrophoresis was carried out for 30 min at 21 V in electrophoresis solution (pH >13). Slides were then stained with SYBR™ Gold (Thermo Fisher Scientific). Tail DNA content was analyzed with Comet score 1.5 software. DNA strand breakage was expressed as “comet tail moment”. The tail moment was measured for a minimum of 50 cells per sample, and average damage from 3 independent experiments was calculated.

Clonogenic Assay: Five thousand cells were seeded in triplicate into six-well plates and allowed to adhere overnight. Cells were then cultured in absence or presence of drug for 7-10 days as indicated. Remaining cells were fixed with formaldehyde (4%), stained with Crystal violet solution (sigma), and photographed using a digital scanner.

Chromatin Immunoprecipitation (ChIP-qPCR): ChIP assays were performed with EZ-Magna ChIP™ A/G Chromatin Immunoprecipitation Kit (Millipore Corp) as described in manufacturer's instructions. Briefly, cells were crosslinked with 1% formaldehyde. After cell lysis, isolated nuclei were subjected to sonication for chromatin fragmentation. Sheared chromatin was diluted in diluted buffer, and divided into aliquots for immunoprecipitation. Anti-BRD4 antibody (1:50, #13440S, Cell Signaling), anti-H3K27ac antibody (1:100, ab4729, Abcam), anti-H3K4M1 antibody (1:200, ab8895, Abcam), anti-Pol II antibody (1:100, sc-47701, Santa Cruz) or normal Rabbit IgG control (1:200, #2729, Cell Signaling) were added to chromatin samples, followed by overnight incubation at 4° C., with rotation. Antibody-chromatin complexes were captured using magnetic protein A/G beads. Purified DNAs were subjected to quantitative PCR (qPCR). All primers are list in Table 2.

Detection of ssDNA by Immunofluorescence: Cells were grown in 50 mg/ml BrdU for two doubling times before irradiation. Where indicated, 200 nM JQ1 was added 4 hr before irradiation. Cells were placed on ice 10 min before irradiation and kept on ice during the irradiation with 10 Gy. Warm media with or without JQ1 was added for 4 hr at 37° C. BrdU was stained (anti-BrdU, ab8152, 1:100 from Abcam) in non-denaturing conditions which enables detection of BrdU incorporated in ssDNA. TE-2000 imaging acquisition system (Nikon) equipped with a 60× objective lens was used to capture images. Stained was quantified by ImageJ.

Immunofluorescence Staining and Microscopy: Briefly, cells were washed with PBS and fixed with 4% paraformaldehyde for 5 min, followed by permeabilization with 0.5% NP-40 and 1% Triton X-100 for 10 min Cells were then blocked with 5% FBS for 30 min and incubated with primary antibody (anti-RAD51, PC130, 1:100; anti-g-H2AX (Ser139), clone JBW301, 1:1000 from Millipore Corp; anti-RPA32, ab2175, 1:500 from Abcam) for 2 hr, followed by secondary antibody incubation for 1 hr at room temperature. Slides were sealed in mounting medium containing DAPI (Vector Laboratories, H1200) for further image acquisition. TE-2000 imaging acquisition system (Nikon) equipped with a 60× objective lens was used to capture images. Stained was quantified by ImageJ.

HR Repair Analysis: U2OS DR-GFP cells contain a single copy of the HR repair reporter substrate DR-GFP, which contains two nonfunctional GFP open reading frames, including one GFP-coding sequence that is interrupted by a recognition site for the I-SceI endonuclease. Expression of I-SceI leads to formation of a DSB in the I-SceI GFP allele, which can be repaired by HR using the nearby GFP sequence lacking the N- and C-termini, thereby producing functional GFP that can be detected by flow cytometry. To examine the role of JQ1 or individual genes in DSB repair, cells were treated with JQ1 (100 nM), AZD5153 (100 nM) or transfected with CtIP or BRD4 siRNA for 24 hr. Then, cells were transfected with a plasmid expressing I-SceI (pCBASce) for 48 hr. Cells transfected with an empty vector were used as a negative control. GFP-expressing plasmid (pEGFP-C1) was used for transfection efficiency control. Flow cytometry analysis was performed to detect GFP⁺ cells using FACScalibur with CellQuest software (Becton Dickinson). The repair efficiency was scored as the percentage of GFP⁺ cells.

In Vivo Drug Studies:

OVCAR8 Xenografts: 3×10⁶ OVCAR8 cells were injected s.c. into mouse flanks in a 1:1 mix of PBS and Matrigel. When tumors reached 50 to 200 mm³, drugs were administered daily by [vehicle (0.5% hydroxypropylmethylcellulose and 0.2% Tween 80, oral gavage), BMN673 (0.333 mg/kg, oral gavage), and JQ1 (40 mg/kg, I.P.), or combinations of BMN673 and JQ1, n=6 per group]. Mice were treated for 28 day and sacrificed for tissue analysis. Tumor volumes were calculated using volume=length*width/2.

WU-BC3 PDX: 3×10⁶ WU-BC3 cells (Ma et al., 2012) were injected subcutaneously into flanks mice in a 1:1 mix of PBS and Matrigel. After palpable tumors formed, drugs were administered daily by [vehicle (0.5% hydroxypropylmethylcellulose and 0.2% Tween 80, oral gavage), BMN673 (0.333 mg/kg, oral gavage), and JQ1 (40 mg/kg, I.P), or combinations of BMN673 and JQ1, n=6 per group]. Mice were treated until Day 28 and sacrificed for tissue harvest.

PATX53 PDX: Minced fresh tumor tissue (0.1-0.2 cm³ per mouse) was transplanted subcutaneously into flanks of mice. After palpable tumors formed, drugs were administered daily by [vehicle (0.5% hydroxypropylmethylcellulose and 0.2% Tween 80, oral gavage), Olaparib (100 mg/kg, oral gavage), AZD5153 (2.5 mg/kg, oral gavage), or combinations of Olaparib and AZD5153, n=6 per group]. Mice were treated until Day 28 and sacrificed for tissue harvest.

LPAJ-T127 Syngeneic Breast Cancer Models: LPA-T127 is a primary invasive and metastatic mammary cancer from transgenic mice, with expression of LPA1 receptor in mammary epithelium and a spontaneous KRAS^{Q61H} mutation (Liu et al., 2009; Federico et al., 2017). Minced fresh tumor tissue (0.1-0.2 cm³ per mouse) was transplanted into mammary fat pads of FVB mice. After palpable tumors formed, drugs were administered daily by [vehicle (0.5% hydroxypropylmethylcellulose and 0.2% Tween 80, oral gavage), BMN673 (0.333 mg/kg, oral gavage), and JQ1 (40 mg/kg, I.P.), or combinations of BMN673 and JQ1, n=6 per group]. Mice were sacrificed when tumor diameter reach maximum limit of 2.5 cm at Day 22.

LPA-T127 was also repeated with daily [vehicle (0.5% hydroxypropylmethylcellulose and 0.2% Tween 80, oral gavage), Olaparib (100 mg/kg, oral gavage), AZD5153 (2.5 mg/kg, oral gavage), or combinations of Olaparib and AZD5153, n=6 per group]. Mice were sacrificed when tumor diameter reached maximum limits of 2.5 cmat Day 22 for tissue harvest and blood collection 3 hr after the final treatment.

OVCAR3 Xenograft: 2×3 10⁷ OVCAR3 cells were injected subcutaneously in the right flank of mice. Mice were randomized based on tumor volumes using stratified sampling and enrolled into control and treatment groups. Dosing began when mean tumor size reached approximately 200 mm³ and continued for 35 days. Drugs were administered daily by [vehicle (0.5% hydroxypropylmethylcellulose and 0.2% Tween 80, oral gavage), oral gavage), Olaparib (100 mg/kg, oral gavage), AZD5153 (2.5 mg/kg, oral gavage), or combinations of Olaparib and AZD5153], n=10 per group].

Quantification and statistical analysis: Two-sided Student's t test was used to compare differences between two groups of cells in vitro. Data are presented as means±SEM, and p<0.05 is considered significant. The correlation between groups was determined by Pearson's correlation test. Analysis of variance was used to compare differences among multiple groups. All statistical analyses were done using SPSS 17.0 (SPSS Inc.). Data were analyzed and plotted using GraphPad Prism 6 software and Microsoft Excel.

Data and software availability: Following GEO datasets of BRD4 inhibition were used for gene expression analysis: GSE29799, GSE66048, GSE44929, GSE85840, GSE31365, and GSE43392. CHIP-seq data after treatment with JQ1 with BRD4 antibody were obtained from GSE63581.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ambrosini, et al., Oncotarget 6, 33397-33409.
Asangani, et al. Nature 510, 278-282, 2014.
Broderick, et al., Nat. Cell Biol. 18, 271-280, 2016.
Bryant, et al., Nature 434, 913-917, 2005.
Bunting, et al., Cell 141, 243-254, 2010.
Byers, et al., Cancer Discov. 2, 798-811, 2012.
Cancer Genome Atlas Research Network. Nature 474, 609-615, 2011.
Davies, et al., Nat. Struct. Mol. Biol. 22, 150-157, 2015.
Dawson, et al., Nature 478, 529-533, 2011.
De Santa, et al., PLoS Biol. 8, e1000384, 2010.
Delmore, et al., Cell 146, 904-917, 2011.
Farmer, et al., Nature 434, 917-921, 2005.
Federico, et al., Sci. Adv. 3, e1600957, 2017.
Filippakopoulos, et al., Nature 468, 1067-1073, 2010.
Gosmini, et al., J. Med. Chem. 57, 8111-8131, 2014.
Hoeijmakers, Nature 411, 366-374, 2011.
Huertas, and Jackson, J. Biol. Chem. 284, 9558-9565, 2009.
Huertas, Nat. Struct. Mol. Biol. 17, 11-16, 2010.
Ira, et al., Nature 431, 1011-1017, 2004.
Jackson, and Bartek, Nature 461, 1071-1078, 2009.
Jaspers, et al., Cancer Discov. 3, 68-81, 2013.
Kaidi, et al., Science 329, 1348-1353, 2010.
Kaufman, et al., J. Clin. Oncol. 33, 244-250, 2015.
Kim, et al., Nature 465, 182-187, 2010.
Kondrashova, et al., Cancer Discov. 7, 984-998, 2017.
Ledermann, et al., Lancet Oncol. 17, 1579-1589, 2016.
Li, et al., Cell Rep. 4, 1116-1130, 2013.
Lin, et al., Mol. Cancer Res. 12, 381-393, 2014.
Liu, et al., Cancer Cell 15, 539-550, 2009.
Love′ n, et al., Cell 153, 320-334, 2013.
Ma, et al., J. Clin. Invest. 122, 1541-1552, 2012.
Mirza, et al., N. Engl. J. Med. 375, 2154-2164, 2016.
Murai, et al., Cancer Res. 72, 5588-5599, 2012.
Nicodeme, et al., Nature 468, 1119-1123, 2010.
Norquist, et al., J. Clin. Oncol. 29, 3008-3015, 2011.
Orthwein, et al., Nature 528, 422-426, 2015.
Peng, et al., Nat. Commun. 5, 3361, 2014.
Polato, et al., J. Exp. Med. 211, 1027-1036, 2014.
Puissant, et al., Cancer Discov. 3, 308-323, 2013.
Ray Chaudhuri, et al., Nature 535, 382-387, 2016.
Rhyasen, et al., Mol. Cancer Ther. 15, 2563-2574, 2016.
Sartori, et al., Nature 450, 509-514, 2007.
Shu, et al., Nature 529, 413-417, 2016.
Sun, et al., Sci. Transl. Med. 9, 392, 2017.
Swisher, et al., Lancet Oncol. 18, 75-87, 2017.
Wang, et al., Oncotarget 7, 7701-7714, 2016.
Xu, et al., Nature 521, 541-544, 2015.
Yang, et al., Cell 19, 535-545, 2005.
Yang, et al., Sci. Transl. Med. 9, 400, 2017.
Yeo, et al., Hum. Mol. Genet. 23, 3695-3705, 2014.
Yokoyama, et al., Nature 459, 460-463, 2009.
Zhang, et al., Theranostics 6, 219-230, 2016.
Zuber, et al., Nature 478, 524-528, 2011.

1. A method for treating cancer in a subject comprising administering an effective amount of a poly-ADP-ribose polymerase (PARP) inhibitor in combination with a bromodomain-containing protein 4 (BRD4) inhibitor to the subject.

2. The method of claim 1, wherein the administration of the PARP inhibitor and BRD4 inhibitor results in greater reduction in tumor growth or greater reduction in tumor mass relative to administration of PARP inhibitor or BRD4 inhibitor alone.

3. The method of claim 1, wherein the subject is PARP inhibitor resistant.

4. The method of claim 1, wherein the subject is PARP inhibitor sensitive.

5. The method of claim 4, wherein the administration of the PARP inhibitor in combination with the BRD4 inhibitor prevents emergence of PARP inhibitor resistance.

6. The method of claim 1, wherein the subject is human.

7. The method of claim 1, wherein the cancer is a RAS/BRAF, BRCA1/2, and/or p53 mutant cancer.

8. The method of claim 7, wherein the RAS/BRAF mutation is KRAS or NRAS.

9. The method of claim 1, wherein the cancer is homologous recombination (HR) competent.

10. The method of claim 9, wherein the HR competent cancer is a RAS/BRAF, BRCA1/2, and/or p53 wild-type cancer.

11. The method of any of claims 1-10, wherein the cancer is breast cancer, ovarian cancer, pancreatic cancer, colorectal cancer, lung cancer, or melanoma.

12. The method of any of claims 1-10, wherein the subject has increased expression of C-terminal binding protein interacting protein (CtIP).

13. The method of any of claims 1-10, wherein the PARP inhibitor is Olaparib, BMN673, Niraparib, Rucaparib, or ABT888 (Veliparab).

14. The method of any of claims 1-10, wherein the PARP inhibitor is BMN673.

15. The method of any of claims 1-10, wherein the BRD4 inhibitor is JQ1, GSK1210151A (I-BET151), GSK1324726A (I-BET-726), or AZD5153.

16. The method of any of claims 1-10, wherein the BRD4 inhibitor is JQ1.

17. The method of claim 1, wherein the PARP inhibitor and/or BRD4 inhibitor are administered orally, intravenously, intraperitoneally, intratracheally, intratumorally, intramuscularly, endoscopically, intralesionally, percutaneously, subcutaneously, regionally, or by direct injection or perfusion.

18. The method of claim 1, wherein the PARP inhibitor and/or BRD4 inhibitor are administered orally.

19. The method of claim 18, wherein the PARP inhibitor is administered at a dose of 200-400 mg/day.

20. The method of claim 18, wherein the BRD4 inhibitor is administered at a dose of 10-40 mg/day.

21. The method of claim 1, wherein the PARP inhibitor and BRD4 inhibitor are administered more than once.

22. The method of claim 1, wherein the PARP inhibitor and BRD4 inhibitor are administered daily.

23. The method of claim 1, wherein the PARP inhibitor and BRD4 inhibitor are administered concurrently.

24. The method of claim 1, wherein the PARP inhibitor is administered before the BRD4 inhibitor.

25. The method of claim 1, wherein the BRD4 inhibitor is administered before the PARP inhibitor.

26. The method of claim 1, wherein the administration results in induction of homologous repair deficiency.

27. The method of claim 26, wherein the induction of homologous repair deficiency results in an increase in DNA damage and checkpoint defects.

28. The method of claim 1, wherein the administration results decreased expression of WEE1 and/or TOPBP1.

29. The method of claim 1, wherein the administration results in decreased expression of C-terminal binding protein interacting protein (CtIP).

30. The method of claim 1, further comprising the step of administering at least one additional therapeutic agent to the subject.

31. The method of claim 1, wherein the subject receives at least one additional type of therapy.

32. The method of claim 31, wherein the at least one additional type of therapy is selected from the group consisting of chemotherapy, radiotherapy, targeted therapy, and immunotherapy.

33. A method for treating a PARP-resistant cancer or preventing PARP resistance in a subject comprising administering an effective amount of a BRD4 inhibitor to the subject.

34. The method of claim 33, wherein BRD4 inhibition resensitizes PARP resistant cells to PARP inhibition.

35. The method of claim 34, further comprising administering an effective amount of a PARP inhibitor to the subject.

36. The method of claim 35, wherein the cancer is breast cancer, ovarian cancer, pancreatic cancer, colorectal cancer, lung cancer, or melanoma.

37. The method of claim 35, wherein the subject has increased expression of C-terminal binding protein interacting protein (CtIP).

38. The method of claim 35, wherein the PARP inhibitor is Olaparib, BMN673, Niraparib, Rucaparib, or ABT888 (Veliparab).

39. The method of claim 35, wherein the PARP inhibitor is BMN673.

40. The method of claim 35, wherein the BRD4 inhibitor is JQ1, GSK1210151A (I-BET151), GSK1324726A (I-BET-726), or AZD5153.

41. The method of claim 35, wherein the BRD4 inhibitor is JQ1.

42. The method of claim 35, wherein the PARP inhibitor and/or BRD4 inhibitor are administered orally, intravenously, intraperitoneally, intratracheally, intratumorally, intramuscularly, endoscopically, intralesionally, percutaneously, subcutaneously, regionally, or by direct injection or perfusion.

43. The method of claim 35, wherein the PARP inhibitor and/or BRD4 inhibitor are administered intravenously.

44. The method of claim 35, wherein the PARP inhibitor and BRD4 inhibitor are administered more than once.

45. The method of claim 35, wherein the PARP inhibitor and BRD4 inhibitor are administered daily.

46. The method of claim 35, wherein the PARP inhibitor and BRD4 inhibitor are administered concurrently.

47. The method of claim 35, wherein the PARP inhibitor is administered before the BRD4 inhibitor.

48. The method of claim 35, wherein the BRD4 inhibitor is administered before the PARP inhibitor.

49. The method of claim 35, wherein the administration results decreased expression of WEE1 and/or TOPBP1.

50. The method of claim 35 wherein the administration results in decreased expression of C-terminal binding protein interacting protein (CtIP).

51. The method of claim 35, further comprising the step of administering at least one additional therapeutic agent to the subject.

52. The method of claim 35, wherein the subject receives at least one additional type of therapy.

53. The method of claim 35, wherein the at least one additional type of therapy is selected from the group consisting of chemotherapy, radiotherapy, and immunotherapy.

54. A method of predicting response to a PARP inhibitor comprising measuring the expression of CtIP in said subject, wherein low CtIP expression identifies a PARP sensitive cancer and high CtIP expression identifies a PARP resistant cancer.

55. The method of claim 54, wherein a subject with the PARP sensitive cancer is administered an effective amount of a PARP inhibitor.

56. The method of claim 54, wherein a subject with the PARP resistant cancer is administered an effective amount of a BRD4 inhibitor to induce PARP sensitivity.

57. The method of claim 56, wherein said subject is further administered an effective amount of a PARP inhibitor.

58. The method of claim 54, wherein the cancer is breast cancer, ovarian cancer, pancreatic cancer, colorectal cancer, lung cancer, or melanoma.

59. The method of claim 55 or 57, wherein the PARP inhibitor is Olaparib, BMN673, Niraparib, Rucaparib, or ABT888 (Veliparab).

60. The method of claim 55 or 57, wherein the PARP inhibitor is BMN673.

61. The method of claim 56, wherein the BRD4 inhibitor is JQ1, GSK1210151A (I-BET151), GSK1324726A (I-BET-726), or AZD5153.

62. The method of claim 56, wherein the BRD4 inhibitor is JQ1.

63. The method of any of claims 55-57, wherein the PARP inhibitor and/or BRD4 inhibitor are administered orally, intravenously, intraperitoneally, intratracheally, intratumorally, intramuscularly, endoscopically, intralesionally, percutaneously, subcutaneously, regionally, or by direct injection or perfusion.

64. The method of any of claims 55-57, wherein the PARP inhibitor and/or BRD4 inhibitor are administered intravenously.

65. The method of any of claims 55-57, wherein the PARP inhibitor and BRD4 inhibitor are administered more than once.

66. The method of any of claims 55-57, wherein the PARP inhibitor and BRD4 inhibitor are administered daily.

67. The method of any of claims 55-57, wherein the PARP inhibitor and BRD4 inhibitor are administered concurrently.

68. The method of any of claims 55-57, wherein the PARP inhibitor is administered before the BRD4 inhibitor.

69. The method of any of claims 55-57, wherein the BRD4 inhibitor is administered before the PARP inhibitor.

70. The method of any of claims 55-57, further comprising the step of administering at least one additional therapeutic agent to the subject.

71. The method of any of claims 55-57, wherein the subject receives at least one additional type of therapy.

72. The method of any of claims 55-57, wherein the at least one additional type of therapy is selected from the group consisting of chemotherapy, radiotherapy, targeted therapy, and immunotherapy.

73. A method of treating cancer in a subject comprising administering a BRD4 inhibitor to the subject, wherein the patient has been determined to be resistant to PARP inhibitors.

74. The method of claim 54, wherein the cancer is breast cancer, ovarian cancer, pancreatic cancer, colorectal cancer, lung cancer, or melanoma.

75. The method of claim 55 or 57, wherein the PARP inhibitor is Olaparib, BMN673, Niraparib, Rucaparib, or ABT888 (Veliparab).

76. The method of claim 56, wherein the BRD4 inhibitor is JQ1, GSK1210151A (I-BET151), GSK1324726A (I-BET-726), or AZD5153.

77. A method of inhibiting CtIP expression in a subject comprising administering an effective amount of BRD4 inhibitor to said subject.

78. The method of claim 77, wherein the subject has cancer.

79. The method of claim 78, wherein the cancer is breast cancer, ovarian cancer, pancreatic cancer, colorectal cancer, non-small cell lung cancer, or melanoma.

80. The method of claim 77, wherein the BRD4 inhibitor is JQ1, GSK1210151A (I-BET151), GSK1324726A (I-BET-726), or AZD5153.

81. The method of claim 77, further comprising administering an effective amount of a PARP inhibitor to the subject.

82. The method of claim 81, wherein the PARP inhibitor is Olaparib, BMN673, Niraparib, Rucaparib, or ABT888 (Veliparab).

83. A pharmaceutical composition comprising a PARP inhibitor and a BRD4 inhibitor.

84. The pharmaceutical composition of claim 83 for use in the treatment of cancer.

85. The use of a therapeutically effective amount of a PARP inhibitor and a BRD4 inhibitor for the treatment of cancer.

86. The use of claim 85, wherein the cancer is breast cancer, ovarian cancer, pancreatic cancer, colorectal cancer, lung cancer, or melanoma.

87. The use of claim 85, wherein the BRD4 inhibitor is JQ1, GSK1210151A (I-BET151), GSK1324726A (I-BET-726), or AZD5153.

88. The use of claim 85, wherein the PARP inhibitor is Olaparib, BMN673, Niraparib, Rucaparib, or ABT888 (Veliparab).

89. A composition comprising a therapeutically effective amount of a PARP inhibitor and a BRD4 inhibitor for the treatment of cancer in a subject.

90. Use of a PARP inhibitor and a BRD4 inhibitor in the manufacture of a medicament for the treatment of cancer.

91. The use of claim 90, wherein the PARP inhibitor is Olaparib, BMN673, Niraparib, Rucaparib, or ABT888 (Veliparab).

92. The use of claim 91, wherein the BRD4 inhibitor is JQ1, GSK1210151A (I-BET151), GSK1324726A (I-BET-726), or AZD5153.

93. The use of claim 90, wherein the cancer is breast cancer, ovarian cancer, pancreatic cancer, colorectal cancer, lung cancer, or melanoma.
